SEQUENCE LISTING (4 sequences)

SEQ ID NO: 1 - protein, 1740 aa, Homo sapiens
MADEEAGGTERMEISAELPQTPQRLASWWDQQVDFYTAFLHHLAQLVPEIYFAEMDPDLEKQEESVQMSIFTPLEWYLFGEDPDICLEKLKHSGAFQLCGRVFKSGETTYSCRDCAIDPTCVLCMDCFQDSVHKNHRYKMHTSTGGGFCDCGDTEAWKTGPFCVNHEPGRAGTIKENSRCPLNEEVIVQARKIFPSVIKYVVEMTIWEEEKELPPELQIREKNERYYCVLFNDEHHSYDHVIYSLQRALDCELAEAQLHTTAIDKEGRRAVKAGAYAACQEAKEDIKSHSENVSQHPLHVEVLHSEIMAHQKFALRLGSWMNKIMSYSSDFRQIFCQACLREEPDSENPCLISRLMLWDAKLYKGARKILHELIFSSFFMEMEYKKLFAMEFVKYYKQLQKEYISDDHDRSISITALSVQMFTVPTLARHLIEEQNVISVITETLLEVLPEYLDRNNKFNFQGYSQDKLGRVYAVICDLKYILISKPTIWTERLRMQFLEGFRSFLKILTCMQGMEEIRRQVGQHIEVDPDWEAAIAIQMQLKNILLMFQEWCACDEELLLVAYKECHKAVMRCSTSFISSSKTVVQSCGHSLETKSYRVSEDLVSIHLPLSRTLAGLHVRLSRLGAVSRLHEFVSFEDFQVEVLVEYPLRCLVLVAQVVAEMWRRNGLSLISQVFYYQDVKCREEMYDKDIIMLQIGASLMDPNKFLLLVLQRYELAEAFNKTISTKDQDLIKQYNTLIEEMLQVLIYIVGERYVPGVGNVTKEEVTMREIIHLLCIEPMPHSAIAKNLPENENNETGLENVINKVATFKKPGVSGHGVYELKDESLKDFNMYFYHYSKTQHSKAEHMQKKRRKQENKDEALPPPPPPEFCPAFSKVINLLNCDIMMYILRTVFERAIDTDSNLWTEGMLQMAFHILALGLLEEKQQLQKAPEEEVTFDFYHKASRLGSSAMNIQMLLEKLKGIPQLEGQKDMITWILQMFDTVKRLREKSCLIVATTSGSESIKNDEITHDKEKAERKRKAEAARLHRQKIMAQMSALQKNFIETHKLMYDNTSEMPGKEDSIMEEESTPAVSDYSRIALGPKRGPSVTEKEVLTCILCQEEQEVKIENNAMVLSACVQKSTALTQHRGKPIELSGEALDPLFMDPDLAYGTYTGSCGHVMHAVCWQKYFEAVQLSSQQRIHVDLFDLESGEYLCPLCKSLCNTVIPIIPLQPQKINSENADALAQLLTLARWIQTVLARISGYNIRHAKGENPIPIFFNQGMGDSTLEFHSILSFGVESSIKYSNSIKEMVILFATTIYRIGLKVPPDERDPRVPMLTWSTCAFTIQAIENLLGDEGKPLFGALQNRQHNGLKALMQFAVAQRITCPQVLIQKHLVRLLSVVLPNIKSEDTPCLLSIDLFHVLVGAVLAFPSLYWDDPVDLQPSSVSSSYNHLYLFHLITMAHMLQILLTVDTGLPLAQVQEDSEEAHSASSFFAEISQYTSGSIGCDIPGWYLWVSLKNGITPYLRCAALFFHYLLGVTPPEELHTNSAEGEYSALCSYLSLPTNLFLLFQEYWDTVRPLLQRWCADPALLNCLKQKNTVVRYPRKRNSLIELPDDYSCLLNQASHFRCPRSADDERKHPVLCLFCGAILCSQNICCQEIVNGEEVGACIFHALHCGAGVCIFLKIRECRVVLVEGKARGCAYPAPYLDEYGETDPGLKRGNPLHLSRERYRKLHLVWQQHCIIEEIARSQETNQM

SEQ ID NO: 2 - protein, 1755 aa, Homo sapiens
MASELEPEVQAIDRSLLECSAEEIAGKWLQATDLTREVYQHLAHYVPKIYCRGPNPFPQKEDMLAQHVLLGPMEWYLCGEDPAFGFPKLEQANKPSHLCGRVFKVGEPTYSCRDCAVDPTCVLCMECFLGSIHRDHRYRMTTSGGGGFCDCGDTEAWKEGPYCQKHELNTSEIEEEEDPLVHLSEDVIARTYNIFAITFRYAVEILTWEKESELPADLEMVEKSDTYYCMLFNDEVHTYEQVIYTLQKAVNCTQKEAIGFATTVDRDGRRSVRYGDFQYCEQAKSVIVRNTSRQTKPLKVQVMHSSIVAHQNFGLKLLSWLGSIIGYSDGLRRILCQVGLQEGPDGENSSLVDRLMLSDSKLWKGARSVYHQLFMSSLLMDLKYKKLFAVRFAKNYQQLQRDFMEDDHERAVSVTALSVQFFTAPTLARMLITEENLMSIIIKTFMDHLRHRDAQGRFQFERYTALQAFKFRRVQSLILDLKYVLISKPTEWSDELRQKFLEGFDAFLELLKCMQGMDPITRQVGQHIEMEPEWEAAFTLQMKLTHVISMMQDWCASDEKVLIEAYKKCLAVLMQCHGGYTDGEQPITLSICGHSVETIRYCVSQEKVSIHLPVSRLLAGLHVLLSKSEVAYKFPELLPLSELSPPMLIEHPLRCLVLCAQVHAGMWRRNGFSLVNQIYYYHNVKCRREMFDKDVVMLQTGVSMMDPNHFLMIMLSRFELYQIFSTPDYGKRFSSEITHKDVVQQNNTLIEEMLYLIIMLVGERFSPGVGQVNATDEIKREIIHQLSIKPMAHSELVKSLPEDENKETGMESVIEAVAHFKKPGLTGRGMYELKPECAKEFNLYFYHFSRAEQSKAEEAQRKLKRQNREDTALPPPVLPPFCPLFASLVNILQSDVMLCIMGTILQWAVEHNGYAWSESMLQRVLHLIGMALQEEKQHLENVTEEHVVTFTFTQKISKPGEAPKNSPSILAMLETLQNAPYLEVHKDMIRWILKTFNAVKKMRESSPTSPVAETEGTIMEESSRDKDKAERKRKAEIARLRREKIMAQMSEMQRHFIDENKELFQQTLELDASTSAVLDHSPVASDMTLTALGPAQTQVPEQRQFVTCILCQEEQEVKVESRAMVLAAFVQRSTVLSKNRSKFIQDPEKYDPLFMHPDLSCGTHTSSCGHIMHAHCWQRYFDSVQAKEQRRQQRLRLHTSYDVENGEFLCPLCECLSNTVIPLLLPPRNIFNNRLNFSDQPNLTQWIRTISQQIKALQFLRKEESTPNNASTKNSENVDELQLPEGFRPDFRPKIPYSESIKEMLTTFGTATYKVGLKVHPNEEDPRVPIMCWGSCAYTIQSIERILSDEDKPLFGPLPCRLDDCLRSLTRFAAAHWTVASVSVVQGHFCKLFASLVPNDSHEELPCILDIDMFHLLVGLVLAFPALQCQDFSGISLGTGDLHIFHLVTMAHIIQILLTSCTEENGMDQENPPCEEESAVLALYKTLHQYTGSALKEIPSGWHLWRSVRAGIMPFLKCSALFFHYLNGVPSPPDIQVPGTSHFEHLCSYLSLPNNLICLFQENSEIMNSLIESWCRNSEVKRYLEGERDAIRYPRESNKLINLPEDYSSLINQASNFSCPKSGGDKSRAPTLCLVCGSLLCSQSYCCQTELEGEDVGACTAHTYSCGSGVGIFLRVRECQVLFLAGKTKGCFYSPPYLDDYGETDQGLRRGNPLHLCKERFKKIQKLWHQHSVTEEIGHAQEANQTLVGIDWQHL

SEQ ID NO: 3 - protein, 303 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSQLCGRVFKSGETTYSCRDCAIDPTCVLCMDCFQDSVHKNHRYKMHTSTGGGFCDCGDTEAWKTGPFCVNHEP

SEQ ID NO: 4 - protein, 300 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGSLCGRVFKVGEPTYSCRDCAVDPTCVLCMECFLGSIHRDHRYRMTTSGGGGFCDCGDTEAWKEGPYCQKH

(19) United States
(12) Patent Application Publication
(10) Pub. No.: US 2025/0186562 A1
(43) Pub. Date: Jun. 12, 2025

(54) TARGET SUBSTRATE PROTEIN DEGRADATION PLATFORM

(71) Applicant: The Regents of the University of California, Oakland, CA (US)

(72) Inventors: Yuan Chen, La Jolla, CA (US); Nicole Thomas, San Diego, CA (US); Abdelfattah Faouzi, San Diego, CA (US)

(21) Appl. No.: 18/843,646

(22) PCT Filed: Mar. 8, 2023
(86) PCT No.: PCT/US2023/014835

§ 371 Date: Sep. 3, 2024

Related U.S. Application Data
(60) Provisional application No. 63/317,785, filed on Mar. 8, 2022.

(51) Int. Cl. A61K 38/45; A61K 38/07; C07K 5/10

(52) U.S. Cl. CPC A61K 38/45; A61K 38/07; C07K 5/10; C12Y 203/02

(57) ABSTRACT

The disclosure is directed to peptidomimetic compounds, or pharmaceutically acceptable salts thereof, pharmaceutical compositions comprising such peptidomimetic compounds, and methods for degrading target substrate proteins using such compounds.

### CROSS-REFERENCE TO RELATED APPLICATION

This application claims the priority of U.S. provisional application Ser. No. 63/317,785, filed Mar. 8, 2022, the disclosure of which is incorporated herein by reference in its entirety as if fully set forth herein.

### FEDERAL FUNDING

This technology was made with government support under grant number 5R01CA265410-14 from the National Institutes of Health (NIH). The government has certain rights in the technology.

### INCORPORATION BY REFERENCE OF SEQUENCE LISTING

A Sequence Listing is provided herewith as an xml file, “2315993.xml” created on Mar. 8, 2023, and having a size of 8,612 bytes. The content of the xml file is incorporated by reference herein in its entirety.

### BACKGROUND

Protein degradation by the ubiquitin (Ub) system controls the levels of many intracellular proteins in eukaryotic cells through a process known as ubiquitination. The Ub system is involved in regulating many basic cellular processes, including cell division and differentiation, response to environmental stress, immune responses, DNA repair, and apoptosis. Ubiquitination participates in maintaining the balance of proteins in eukaryotic organisms, serving to rapidly remove misfolded proteins.

Ubiquitination is a form of post-translational modification in which the ubiquitin, a 76-amino acid protein, attaches to a substrate protein. It is a three-step process involving three enzymes: ubiquitin-activating enzyme (E1), ubiquitin-conjugating enzyme (E2), and ubiquitin-protein ligase (E3). Ubiquitin activating enzyme E1 uses an ATP dependent process to establish a thioester bond between the C-terminal carboxyl group of ubiquitin and the cysteine group of E1. The E2 ubiquitin-conjugating enzyme then binds to both the activated ubiquitin and E1 enzyme complex. E2 catalyzes the transfer of ubiquitin from the E1 site to the active site on E2 by way of a transesterification reaction. In the final step, E3 ubiquitination ligase creates an isopeptide bond with a lysine of the substrate protein and the C-terminal glycine of ubiquitin. The result of this cascade of reactions is the formation of a ubiquitin-substrate complex.

Multiple molecules of ubiquitin can be attached to any of the seven lysine residues or the N-terminus of ubiquitin to form ubiquitin chains, resulting in polyubiquitination. Polyubiquitination subsequently leads to the initiation of proteolysis of the substrate protein by serving as the recognition signal for the 26S proteasome.

### SUMMARY

Many target substrate proteins (TSPs) cannot be degraded by current E3 ligase (e.g., Proteolysis Targeting Chimeras (PROTACS)) ligands for reasons including expression of the TSPs in a different cellular compartment from the E3 ligase components, burying of TSP lysine residues within the E3 ligase ternary complex causing failure to ubiquinylate the TSPs, and the limited available E3 ligase ligands limiting the diversity of possible TSP compound structures. Compositions and methods are described herein provide single protein E3 ligases that can target a diversity of TSPs and provide more predictable effects. The E3 ligases are useful for targeting proteins for ubiquitination and subsequent proteolysis through proteasome-mediated degradation. The compositions include bi-functional peptidomimetic compounds that engage a target substrate protein and one or more members of the Ubr family of E3 ligases to ubiquitylate the target substrate protein. The disclosure therefore relates to the use of peptidomimetic compounds to decrease levels of the target substrate protein in vivo, providing therapeutically useful treatments for diseases and disorders, such as cancers, that involve aberrant proteins.

### BRIEF DESCRIPTION OF THE DRAWINGS

The drawings illustrate generally, by way of example, but not by way of limitation, various embodiments discussed herein.

FIGS. 1A-B are illustrations of the mechanism of action of peptidomimetic compounds in targeted protein degradation. FIG. 1A depicts the mechanism of action of peptidomimetic compounds that include a linker bound to a ligand for a target substrate protein. FIG. 1B depicts the mechanism of action of peptidomimetic compounds bound to a ligand for a target substrate protein without a linker.

FIG. 2 is an image of an SDS-PAGE gel showing eluted fractions of purified GST-fused Ubr-Box protein.

FIG. 3 depicts chemical structures in a solid-phase peptide synthesis (SPPS) method for synthesizing tripeptide peptidomimetic compounds that bind Ubr-box protein.

FIGS. 4A-C are biophysical assays measuring binding affinity of peptidomimetic compounds for the Ubr-box of Ubr1. FIG. 4A is a graph of a fluorescence polarization (FP) assay showing titration of GST-Ubr-box of Ubr1 or GST into a 9 nM tracer. FIG. 4B is a graph of a competition FP assay showing variations of the binding affinities of peptidomimetic compounds from Table 1.

FIG. 4C are Isothermal Titration Calorimetry (ITC) measurements confirmed the FP measurements.

FIG. 5 are microscopy images show a peptidomimetic compound at two different concentrations blocking R-GFP degradation in cells. The upper panels show fluorescent imaging of the cells and the lower panel show a bright field of the cells.

FIGS. 6A-B depict an example of the peptidomimetic compound linked to a ligand targeting a target substrate protein, ERRa. FIG. 6A depicts chemical reactions for a method of synthesizing a peptidomimetic compound linked to a ligand for targeting ERRa. FIG. 6B is an image of an SDS-PAGE gel showing the reduction of levels of the ERRa protein in cells upon addition of the peptidomimetic compound.

FIG. 7 depicts chemical reactions for a method of synthesizing azide-linked peptidomimetics.

FIGS. 8 and 9 depict chemical reactions for a method of synthesizing a peptidomimetic compound linked to a ligand for targeting ERRa (SPPS=solid phase peptide synthesis).

### DETAILED DESCRIPTION

Compositions, methods, and kits are described herein for making and using bifunctional peptidomimetic compounds to bind a target substrate protein and engage one or more members of the Ubr family of E3 ubiquitin-protein ligases (E3 ligases), resulting in degradation of the target substrate protein by the 26S proteasome. E3 ligases, including Ubr ligases, recognize certain N-terminal amino acids that mark proteins for ubiquitin-mediated degradation. The peptidomimetic compounds described herein engage Ubr ligases with higher affinity, specificity, and stability than peptides of natural amino acid residues.

A ubiquitylated substrate protein bears a covalently linked poly-Ub chain and is degraded by the 26S proteasome. The selectivity of ubiquitylation is determined mainly by E3 ligase, a conserved 76-residue protein which recognizes a substrate protein's degradation signal called a degron. An essential determinant of one class of degrons, called N-degrons, is a substrate's destabilizing N-terminal residue. The set of destabilizing residues in a given cell type yields a rule, called the N-end rule, which relates the in vivo half-life of a protein to the identity of its N-terminal residue. An N-degron consists of two major determinants: a destabilizing N-terminal residue of a substrate protein and its internal lysine residue(s). The latter is the site of formation of a substrate-linked polyubiquitin (poly-Ub) chain.

The E3 ligase that recognizes N-degrons are called N-recognins. Examples of mammalian N-recognins are Ubr1 and Ubr2. Ubr1 and Ubr2 recognize two amino acid residues at the N-terminus, with position one favoring arginine, lysine, and histidine, and position two favoring a hydrophobic amino acid residue. Ubr1 and Ubr2 share substrate binding similarities, and such N-terminal residues are not recognized by other Ubr ligases. When bound to the target substrate protein, the peptidomimetic compounds provide a mimic of the two natural N-terminal amino acid residues recognized and engaged by Ubr1 or Ubr2, or both, with approximately 10-fold selectivity for Ubr2 over Ubr1. The peptidomimetic compounds exhibit approximately 100-fold improvement in affinity for binding Ubr2 over natural N-terminal amino acid residues.

Target substrate protein degradation (TPD) using the peptidomimetic compounds described herein provides useful therapeutics to decrease target substrate protein levels through ubiquitin-mediated proteolysis. The peptidomimetic compounds can also be useful as research reagents for target substrate protein knockdown for protein function discovery without the need to alter gene expression, drug efficacy studies, and discovery of protein targets that are degradable even when they are not drugable. Small molecules such as the peptidomimetic compounds can be used in TPD function in a “catalytic” fashion such that substoichiometric nanomolar (nM) drug concentrations can be therapeutically effective.

Currently only a handful of E3 ligases have been engaged for TPD, and many proteins cannot be degraded by proteolysis-targeting chimeras (PROTACs) utilizing these ligases. Of note, both cereblon protein (encoded by the CRBN gene) and Von Hippel-Lindau (VHL) that dominate the TPD field do not possess E3 ligase activity by themselves. Instead, they bind to cullin-really interesting new gene-E3 (cullin-RING-E3) ligases in multi-protein complexes whose activities are regulated by the constitutive photomorphogenesis 9 (COP9) signalosome that controls their post-translational modifications, such as Neddylation. Many target substrate proteins cannot be degraded by utilizing currently liganded E3s, possibly because the expression of the targets may not occur in cells or cellular compartments that also harbor all components of the E3 ligases and their regulatory components. In addition, the geometry of a ternary complex formed using the limited ligases may not position a lysine residue at a location required for target ubiquitination, leading to failure of their degradation. Furthermore, the limited E3 engagers available also limits the diversity of TPD compound structures, leading to potential common off-target effects. Therefore, it is useful to expand the liganded E3 ligases to single protein E3 ligases to enable TPD for more targets and to increase the predictability of the TPD effects.

Unlike CRBN or VHL, the Ubr family of proteins function as E3 ligases by themselves. They contain both the target substrate protein binding domain and RING or Homologous to the E6-AP Carboxyl Terminus (HECT) domain required for E3 functions. They recognize the target substrate proteins through the Ubr-box domain of Ubr ligases. A recent cryogenic electron microscopy (cryo-EM) study has shown that recognition of N-degron by the Ubr-box domain is independent of the rest of the full-length protein. Previous studies and our unpublished data (not shown) suggests that the substrate binding specificity of Ubr1 and Ubr2 are similar but differs from other Ubr E3 ligases. In addition, Ubr2 is primarily localized in the nucleus in human cells while most current TDP engagers utilizes E3 ligase complexes localize in both cytosol and nucleus, such as CRBN and VHL. Thus, Ubr2 can potentially be engaged to degrade nuclear proteins, offering improved specificity over current TPD platforms.

### Target Substrate Protein

While not wishing to be bound by any specific target substrate protein, the target substrate protein can be any protein for which a decrease in the amount of that protein through Ubr protein-mediated ubiquitination and proteosome degradation is desired. For example, the target substrate protein can be an aberrant protein that can be a therapeutically useful treatment for diseases and disorders, such as cancers. The target substrate protein can include cancer driving proteins, such as androgen receptor for prostate cancer, FLT3 for leukemia, mutant KRas for many cancer types, and β-amyloid, α-synuclein, tau, and polyglutamates for neurodegenerative disorders.

### Mechanism

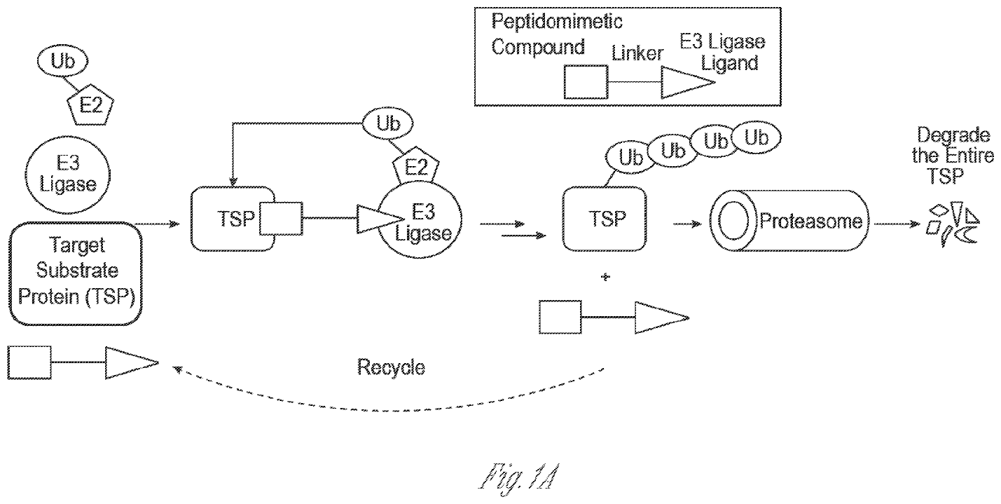

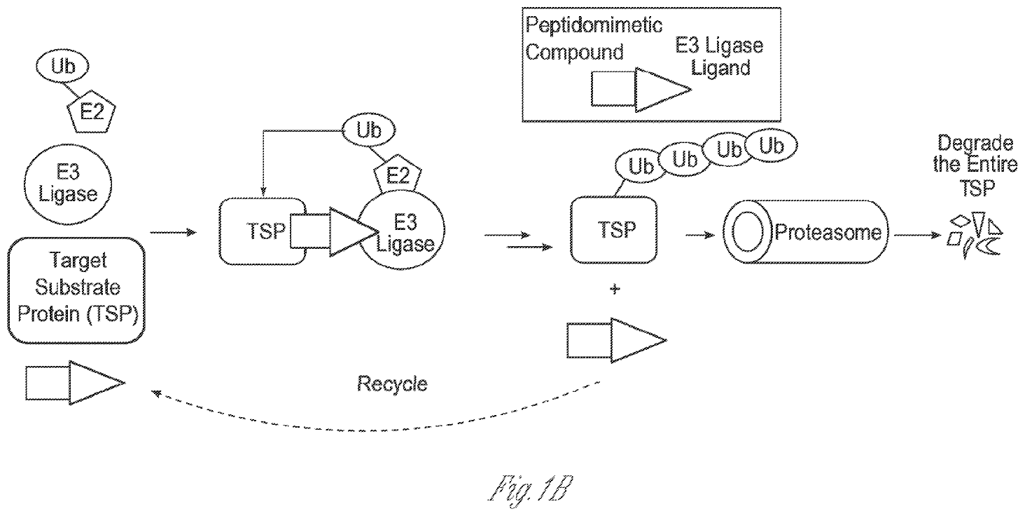

While not wishing to be bound by any specific theory, it is believed that the peptidomimetic compounds described herein act as bi-functional compounds that include a mimic of the two natural amino acids recognized by the E3 ligases Ubr 1 and Ubr2 (FIG. 1A). The peptidomimetic compounds can include a linker that is bound to any suitable ligand for engaging a target substrate protein. The peptidomimetic compounds can also be bound to the ligand for the target substrate protein directly, without a linker (FIG. 1B). Simultaneous binding of the target substrate protein and the Ubr1 or Ubr2 ligase by the peptidomimetic compound induces ubiquitylation of the TSP via engagement of the Ubr1 or Ubr 2 E3 ligase and its association with ubiquitin (Ub) bound E2 ubiquitin-conjugating enzyme to form the ubiquitin-substrate complex. Once the TSP is ubiquitinated, the peptidomimetic compound can be recycled to target another TSP. The ubiquitinated TSP is then degraded by the cellular 26S proteosome. In another example, the peptidomimetic compound may remain bound to the TSP, such as with some peptidomimetic compound that are covalently bound to the TSP and may not be recycled to target another TSP. Peptidomimetic compounds that remain bound to the TSP can be degraded along with the ubiquitinated TSP by the cellular 26S proteosome.

The human Ubr 1 protein is provided as the amino acid sequence of SEQ ID NO. 1:

MADEEAGGTERMEISAELPQTPQRLASWWDQQVDFYTAFLHHLAQLVPEIYFAEMDPDLE
KQEESVQMSIFTPLEWYLFGEDPDICLEKLKHSGAFQLCGRVEKSGETTYSCRDCAIDPT
CVLCMDCFQDSVHKNHRYKMHTSTGGGFCDCGDTEAWKTGPFCVNHEPGRAGTIKENSRC
PLNEEVIVQARKIFPSVIKYVVEMTIWEEEKELPPELQIREKNERYYCVLFNDEHHSYDH
VIYSLQRALDCELAEAQLHTTAIDKEGRRAVKAGAYAACQEAKEDIKSHSENVSQHPLHV
EVLHSEIMAHQKFALRLGSWMNKIMSYSSDFRQIFCQACLREEPDSENPCLISRLMLWDA
KLYKGARKILHELIFSSFFMEMEYKKLFAMEFVKYYKQLQKEYISDDHDRSISITALSVQ
MFTVPTLARHLIEEQNVISVITETLLEVLPEYLDRNNKFNFQGYSQDKLGRVYAVICDLK
YILISKPTIWTERLRMQFLEGERSELKILTCMQGMEEIRRQVGQHIEVDPDWEAAIAIQM
QLKNILLMFQEWCACDEELLLVAYKECHKAVMRCSTSFISSSKTVVQSCGHSLETKSYRV
SEDLVSIHLPLSRTLAGLHVRLSRLGAVSRLHEFVSFEDFQVEVLVEYPLRCLVLVAQVV
AEMWRRNGLSLISQVFYYQDVKCREEMYDKDIIMLQIGASLMDPNKFLLLVLQRYELAEA
FNKTISTKDQDLIKQYNTLIEEMLQVLIYIVGERYVPGVGNVTKEEVTMREIIHLLCIEP
MPHSAIAKNLPENENNETGLENVINKVATFKKPGVSGHGVYELKDESLKDFNMYFYHYSK
TQHSKAEHMQKKRRKQENKDEALPPPPPPEFCPAFSKVINLLNCDIMMYILRTVFERAID
TDSNLWTEGMLQMAFHILALGLLEEKQQLQKAPEEEVTFDFYHKASRLGSSAMNIQMLLE
KLKGIPQLEGQKDMITWILQMEDTVKRLREKSCLIVATTSGSESIKNDEITHDKEKAERK
RKAEAARLHRQKIMAQMSALQKNFIETHKLMYDNTSEMPGKEDSIMEEESTPAVSDYSRI
ALGPKRGPSVTEKEVLTCILCQEEQEVKIENNAMVLSACVQKSTALTQHRGKPIELSGEA
LDPLFMDPDLAYGTYTGSCGHVMHAVCWQKYFEAVQLSSQQRIHVDLEDLESGEYLCPLC
KSLCNTVIPIIPLQPQKINSENADALAQLLTLARWIQTVLARISGYNIRHAKGENPIPIF
FNQGMGDSTLEFHSILSFGVESSIKYSNSIKEMVILFATTIYRIGLKVPPDERDPRVPML
TWSTCAFTIQAIENLLGDEGKPLEGALQNRQHNGLKALMQFAVAQRITCPQVLIQKHLVR
LLSVVLPNIKSEDTPCLLSIDLFHVLVGAVLAFPSLYWDDPVDLQPSSVSSSYNHLYLFH
LITMAHMLQILLTVDTGLPLAQVQEDSEEAHSASSFFAEISQYTSGSIGCDIPGWYLWVS
LKNGITPYLRCAALFFHYLLGVTPPEELHINSAEGEYSALCSYLSLPTNLELLFQEYWDT
VRPLLQRWCADPALLNCLKQKNTVVRYPRKRNSLIELPDDYSCLLNQASHERCPRSADDE
RKHPVLCLFCGAILCSQNICCQEIVNGEEVGACIFHALHCGAGVCIFLKIRECRVVLVEG
KARGCAYPAPYLDEYGETDPGLKRGNPLHLSRERYRKLHLVWQQHCIIEEIARSQETNQM
LFGFNWQLL

The human Ubr 2 protein is provided as the amino acid sequence of SEQ ID NO. 2:

MASELEPEVQAIDRSLLECSAEEIAGKWLQATDLTREVYQHLAHYVPKIYCRGPNPFPQK
EDMLAQHVLLGPMEWYLCGEDPAFGFPKLEQANKPSHLCGRVEKVGEPTYSCRDCAVDPT
CVLCMECELGSIHRDHRYRMTTSGGGGFCDCGDTEAWKEGPYCQKHELNTSEIEEEEDPL
VHLSEDVIARTYNIFAITFRYAVEILTWEKESELPADLEMVEKSDTYYCMLFNDEVHTYE
QVIYTLQKAVNCTQKEAIGFATTVDRDGRRSVRYGDFQYCEQAKSVIVRNTSRQTKPLKV
QVMHSSIVAHQNFGLKLLSWLGSIIGYSDGLRRILCQVGLQEGPDGENSSLVDRLMLSDS
KLWKGARSVYHQLFMSSLLMDLKYKKLFAVRFAKNYQQLQRDEMEDDHERAVSVTALSVQ
FFTAPTLARMLITEENLMSIIIKTEMDHLRHRDAQGRFQFERYTALQAFKERRVQSLILD
LKYVLISKPTEWSDELRQKFLEGFDAFLELLKCMQGMDPITRQVGQHIEMEPEWEAAFTL
QMKLTHVISMMQDWCASDEKVLIEAYKKCLAVLMQCHGGYTDGEQPITLSICGHSVETIR
YCVSQEKVSIHLPVSRLLAGLHVLLSKSEVAYKFPELLPLSELSPPMLIEHPLRCLVLCA
QVHAGMWRRNGFSLVNQIYYYHNVKCRREMFDKDVVMLQTGVSMMDPNHFLMIMLSRFEL
YQIFSTPDYGKRFSSEITHKDVVQQNNTLIEEMLYLIIMLVGERFSPGVGQVNATDEIKR
EIIHQLSIKPMAHSELVKSLPEDENKETGMESVIEAVAHEKKPGLTGRGMYELKPECAKE
FNLYFYHESRAEQSKAEEAQRKLKRQNREDTALPPPVLPPFCPLFASLVNILQSDVMLCI
MGTILQWAVEHNGYAWSESMLQRVLHLIGMALQEEKQHLENVTEEHVVTFTFTQKISKPG
EAPKNSPSILAMLETLQNAPYLEVHKDMIRWILKTFNAVKKMRESSPTSPVAETEGTIME
ESSRDKDKAERKRKAEIARLRREKIMAQMSEMQRHFIDENKELFQQTLELDASTSAVLDH
SPVASDMTLTALGPAQTQVPEQRQFVTCILCQEEQEVKVESRAMVLAAFVQRSTVLSKNR
SKFIQDPEKYDPLFMHPDLSCGTHTSSCGHIMHAHCWQRYFDSVQAKEQRRQQRLRLHTS
YDVENGEFLCPLCECLSNTVIPLLLPPRNIFNNRLNFSDQPNLTQWIRTISQQIKALQFL
RKEESTPNNASTKNSENVDELQLPEGERPDFRPKIPYSESIKEMLTTFGTATYKVGLKVH
PNEEDPRVPIMCWGSCAYTIQSIERILSDEDKPLFGPLPCRLDDCLRSLTRFAAAHWTVA
SVSVVQGHFCKLFASLVPNDSHEELPCILDIDMFHLLVGLVLAFPALQCQDFSGISLGTG
DLHIFHLVTMAHIIQILLTSCTEENGMDQENPPCEEESAVLALYKTLHQYTGSALKEIPS
GWHLWRSVRAGIMPFLKCSALFFHYLNGVPSPPDIQVPGTSHFEHLCSYLSLPNNLICLF
QENSEIMNSLIESWCRNSEVKRYLEGERDAIRYPRESNKLINLPEDYSSLINQASNESCP
KSGGDKSRAPTLCLVCGSLLCSQSYCCQTELEGEDVGACTAHTYSCGSGVGIFLRVRECQ
VLFLAGKTKGCFYSPPYLDDYGETDQGLRRGNPLHLCKERFKKIQKLWHQHSVTEEIGHA
QEANQTLVGIDWQHL

### Peptidomimetic Compounds

The present disclosure provides peptidomimetic compounds of the formulae (I), (II), and (III):

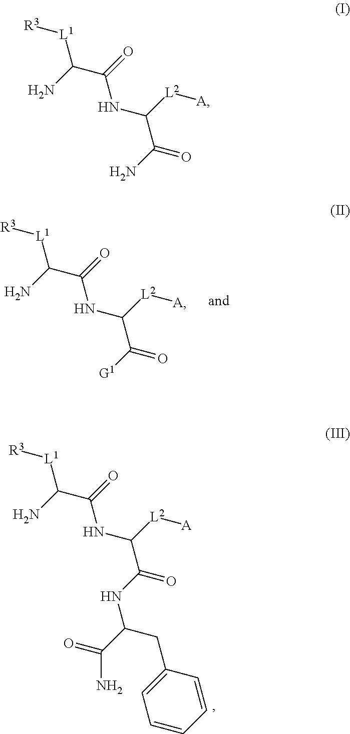

or a pharmaceutically acceptable salt thereof,
wherein:
A is an optionally substituted aryl group, an optionally substituted alkyl group or an optionally substituted heterocyclyl group (e.g., heteroaryl groups, such as thiazole and thiophenyl);
G¹ comprises a moiety that binds to a recognition element on a target substrate protein for degradation through Ubr protein-mediated ubiquitination;
L¹ is a divalent linker (e.g., a divalent alkyl linker or an aryalkyl group);
L² is absent or a divalent alkyl linker; and
R³ comprises an ionizable group, a thiophenyl group or an amide group.

The present disclosure also provides peptidomimetic compounds of formulae (Ia), (IIa), and (IIIa):

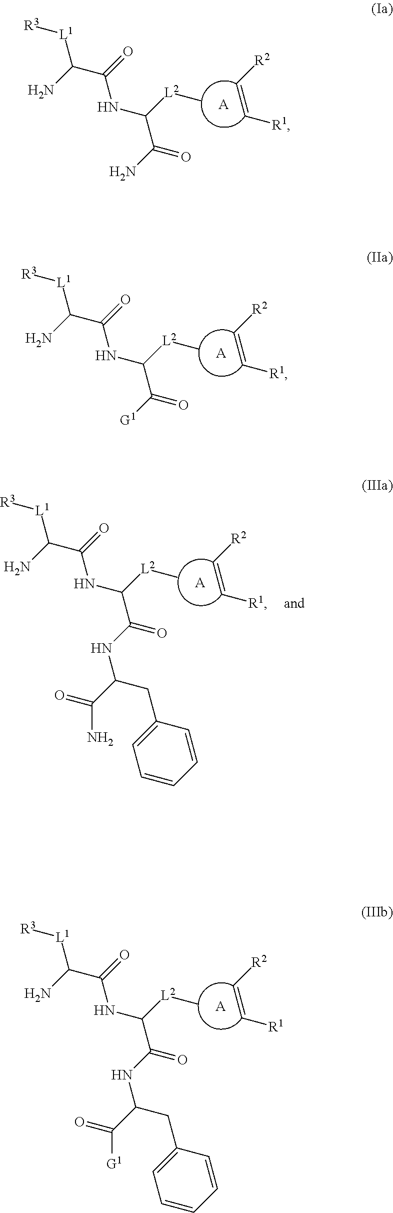

or a pharmaceutically acceptable salt thereof,
wherein:
A, L¹, L², and R³ are defined herein; and
R¹ and R² are each, independently, H or a substituent (e.g., R¹ and R² are each independently NO₂, CN, hydroxy, alkoxy, and halo).

The compounds of the formula (I), (Ia), (II), (IIa), (III), (IIIa), (IIIb) can comprise chiral centers and, as such, can be of the formula (I′), (Ia′), (II′), (IIa′), (III′), (IIIa′), and (IIIb′):

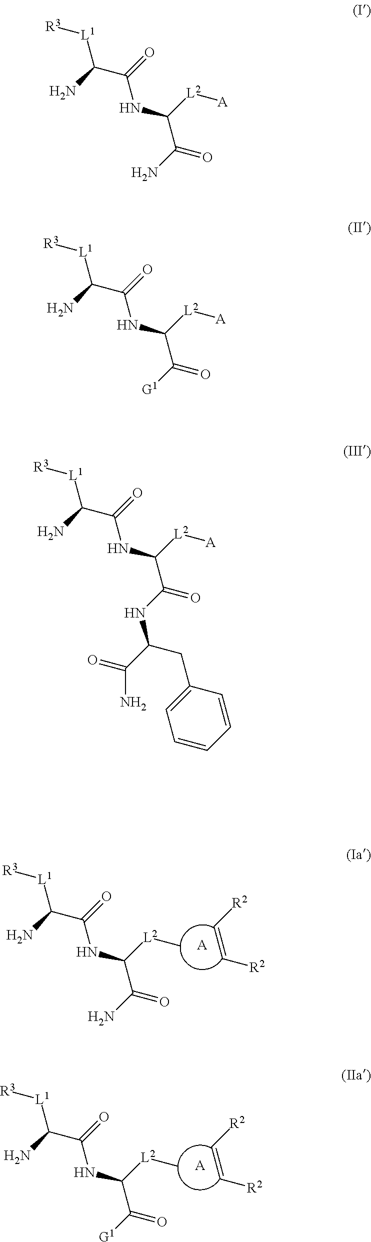

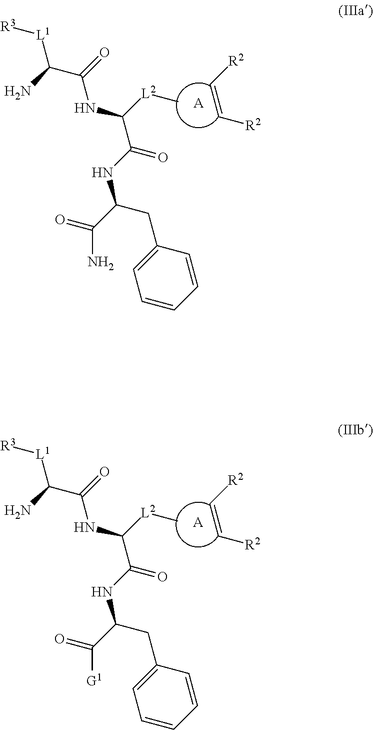

and pharmaceutically acceptable salts thereof.

The disclosure also contemplates compounds of the formula (IV), (IVa), (IVa′), and (IVa″):

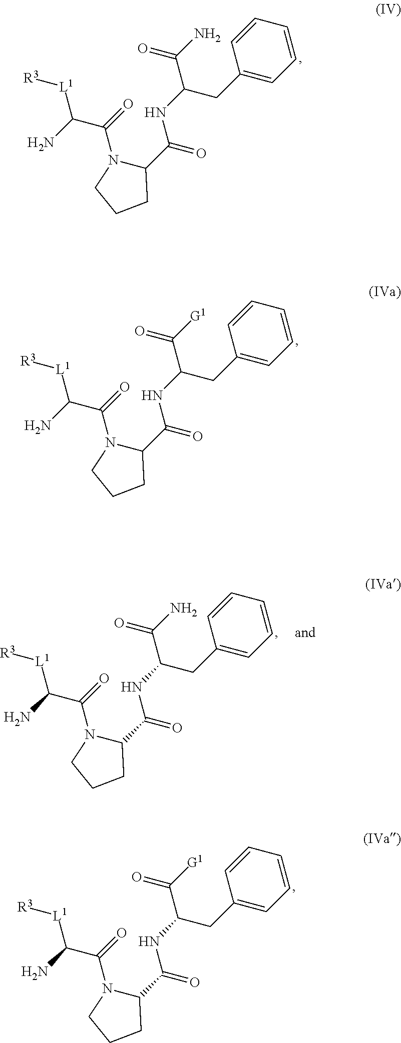

and pharmaceutically acceptable salts thereof.

The moiety that binds to the recognition element on the target substrate protein comprised in the group G¹ can bind to the target substrate protein non-covalently (e.g., via hydrogen bonding) or covalently (e.g., as covalent inhibitors would). A non-limiting example of a moiety that binds to a recognition element on the target substrate protein non-covalently includes a moiety of the formula:

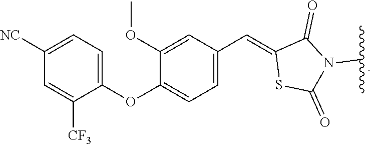 [SMILES: *N1C(=O)S/C(=C\c2ccc(Oc3ccc(C#N)cc3C(F)(F)F)c(OC)c2)C1=O]

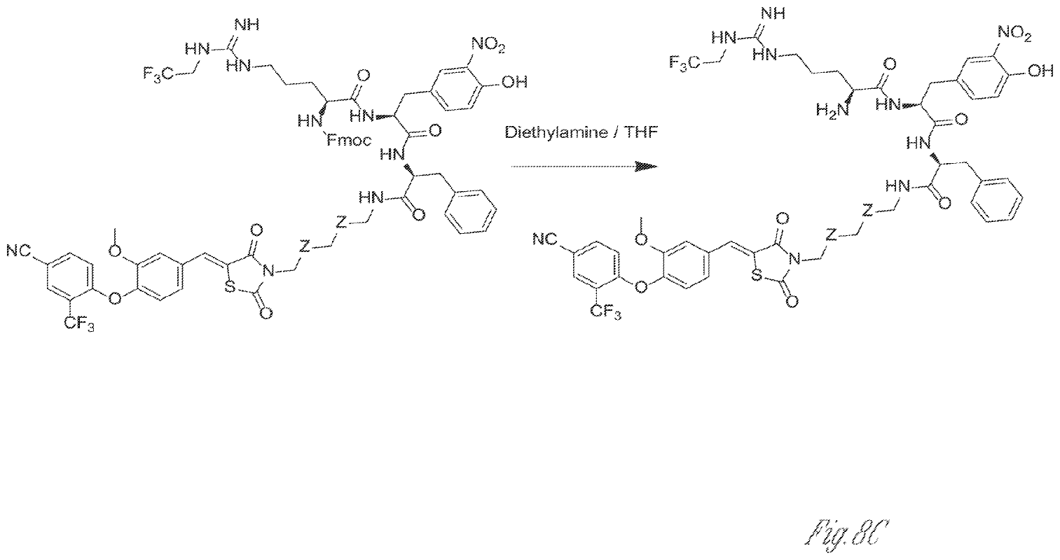

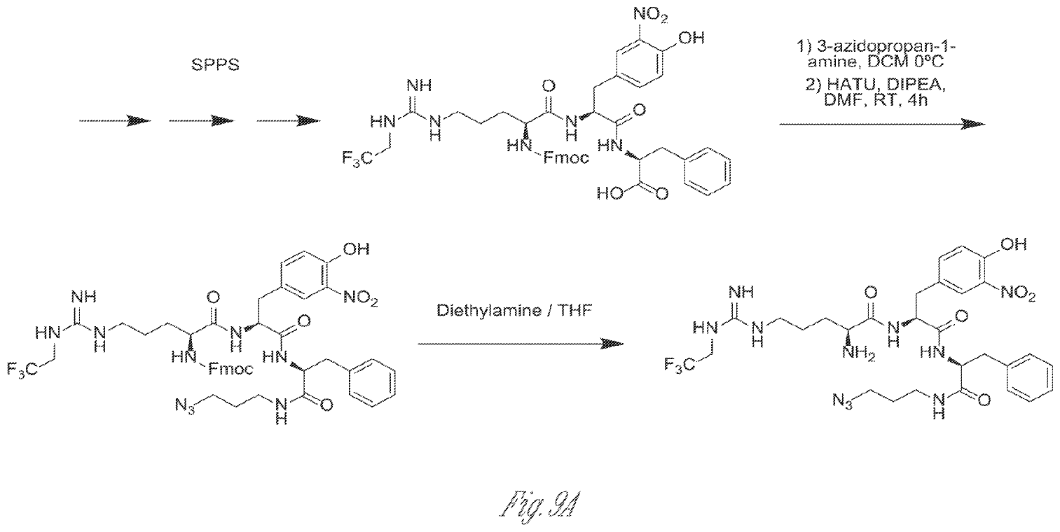

Moieties that bind to a recognition element on a target substrate can be linked directly or indirectly, by way of a divalent linker L³, to the carbonyl group to which G¹ is attached, e.g., in compounds of the formula (II), (IIa), and (IIIb). L³ can, for example, be a divalent alkyl linker, an aryalkyl linking group or L¹. And L³, when present, can be the same or different than L¹. An example of a compound of the formula (IIIb′) is:

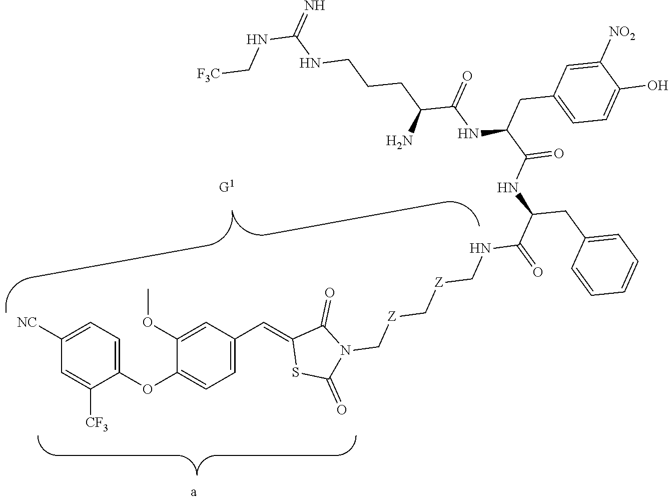

wherein the portion within the bracket (a) is an example of a moiety that binds to a recognition element on a target substrate; and (b) represents the linker L³, wherein Z is CH₂ or O. This molecule, and its synthesis, is also shown in FIG. 8.

In the peptidomimetic compounds of formula (I), (Ia), (II), (IIa), (III), (IIIa), (IIIb), (I′), (Ia′), (II′), (IIa′), (III′), (IIIa′), (IIIb′), (IV), (IVa), (IVa′), and (IVa″), or pharmaceutically acceptable salts thereof, L¹ and L³ (when present), can independently be a divalent alkyl linker including a divalent C₃-C₆-alkyl group, a divalent polyethylene glycol group, one or more amino acids (e.g., phenylalanine) or combinations thereof.

In the peptidomimetic compounds of formula (I), (Ia), (II), (IIa), (III), (IIIa), (IIIb), (I′), (Ia′), (II′), (IIa′), (III′), (IIIa′), (IIIb′), (IV), (IVa), (IVa′), and (IVa″), or pharmaceutically acceptable salts thereof, L¹ and L³ (when present), can independently be any suitable divalent linker comprising acyl, alkyl, cycloalkyl, alkenyl, alkynyl, heterocyclyl or aryl, groups, and combinations thereof (e.g., alkylaryl, cycloalkylene carbonyl, alkyl-S-succinimidyl, and the like), each of which can be optionally substituted and/or interrupted by one or more heteroatoms, such as —O—, —NR⁴—, and —S(O)ₙ— (wherein n is 0, 1 or 2), wherein R⁴ is H or alkyl. Examples of L¹ and L³ (when present) groups include -alkyl-X¹-alkyl- and -alkyl-X¹-alkynyl-linkers, and combinations thereof, wherein X¹ is a bond, alkyl, —O—, —NR⁴—, —S(O)ₙ— or a heterocycle (e.g., a triazole) that can comprise one or more spacer groups, such as alkyl, acyl, amino, and amido groups within the linker or at one or both termini. Thus, for example, L¹ and L³ (when present) can independently be:
-(alkyl-X¹)_{g}-alkyl-, -(alkyl-X¹)_{g}-alkynyl-, —C(O)NR⁴—X¹-(alkyl-X¹)_{g}-alkyl-, —C(O)NR⁴—X¹-(alkyl-X¹)_{g}-alkyl-C(O)—, —C(O)NR⁴—X¹-(alkyl-X¹)_{g}-alkynyl-, —C(O)NR⁴—X¹-(alkyl-X¹)_{g}-alkynyl-C(O)—, —C(O)NR⁴—X¹-(alkyl-X¹)_{g}-alkyl-NR⁴—, —C(O)NR⁴—X¹-(alkyl-X¹)_{g}-alkyl-C(O)—NR⁴—, —C(O)NR⁴—X¹-(alkyl-X¹)_{g}-alkynyl-NR⁴—, —C(O)NR⁴—X¹-(alkyl-X¹)_{g}-alkynyl-C(O)—NR⁴—, —NR⁴—X¹-(alkyl-X¹)_{g}-alkyl-, —NR⁴—X¹-(alkyl-X¹)_{g}-alkyl-C(O)—, —NR⁴—X¹-(alkyl-X¹)_{g}-alkynyl-, —NR⁴—X¹, (alkyl-X¹)_{g}-alkynyl-C(O)—, —NR⁴—X¹-(alkyl-X¹)_{g}-alkyl-NR⁴—, —NR—X¹-(alkyl-X¹)_{g}-alkyl-O—, —NR⁴—X¹-(alkyl-X¹)_{g}-alkyl-C(O)—NR⁴—, —NR⁴—X¹-(alkyl-X¹)_{g}-alkynyl-NR⁴—, —NR⁴—X¹-(alkyl-X¹)_{g}-alkynyl-C(O)—NR⁴—, —C(O)—X¹-(alkyl-X¹)_{g}-alkyl-, —C(O)—X¹-(alkyl-X¹)_{g}-alkyl-C(O)—, —C(O)—X¹-(alkyl-X¹)_{g}-alkynyl-, —C(O)—X¹-(alkyl-X¹)_{g}-alkynyl- —C(O)—, —C(O)—X¹-(alkyl-X¹)_{g}-alkyl-NR⁴—, —C(O)—X¹-(alkyl-X¹)_{g}-alkyl-C(O)—NR⁴—, —C(O)—X¹-(alkyl-X¹)_{g}-alkynyl-NR⁴—, —C(O)—X¹-(alkyl-X¹)_{g}-alkynyl-C(O)—NR⁴—, —NH—X¹—C(O)—X¹-alkylyl- and combinations thereof, wherein g is an integer from 0 to 20 (e.g., 0 to 10, 0 to 5, 1 to 3, 1 to 5, 4 to 10, 2 to 10, 2 to 5 or 3 to 9) and each group (alkyl-X¹) being the same or different. Examples of L¹ and L³ (when present) include:

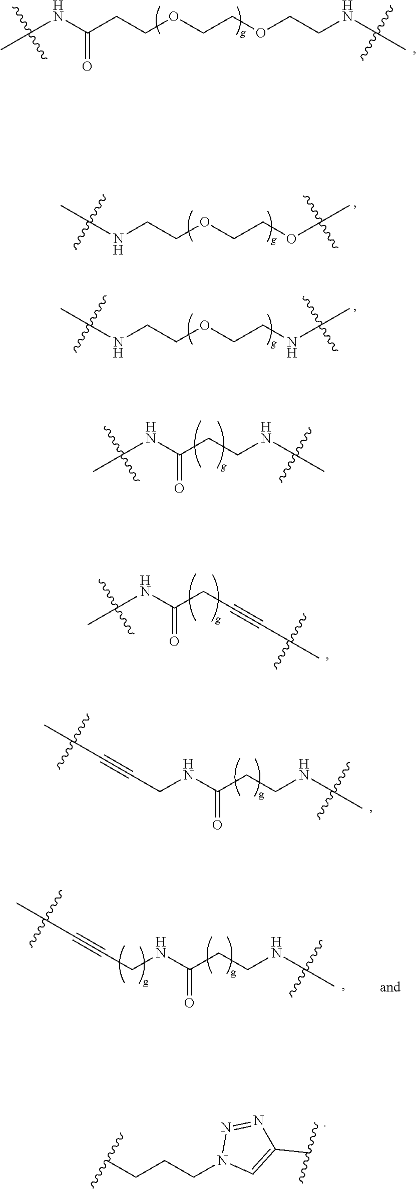

In the peptidomimetic compounds of formula (I), (Ia), (II), (IIa), (III), (IIIa), (IIIb), (I′), (Ia′), (II′), (IIa′), (III′), (IIIa′), (IIIb′), (IV), (IVa), (IVa′), and (IVa″), or pharmaceutically acceptable salts thereof, L¹ and L³ (when present) can further comprise natural amino acids, non-natural amino acids, or a combination thereof. The term “amino acid” as used herein includes molecular fragments or radicals comprising an aminoalkylcarboxylate, where the alkyl radical is optionally substituted with alkyl, hydroxy alkyl, sulfhydrylalkyl, aminoalkyl, carboxyalkyl, and the like, including groups corresponding to the naturally occurring amino acids, such as serine, cysteine, lysine, tryptophan, methionine, aspartic acid, glutamic acid, and the like. The term “non-natural amino acids” includes citrulline, hydroxyproline, norleucine, 3-nitrotyrosine, nitroarginine, ornithine, naphtylalanine, methionine sulfoxide, and methionine sulfone.

In the peptidomimetic compounds of formula (I), (Ia), (II), (IIa), (III), (IIIa), (IIIb), (I′), (Ia′), (II′), (Ila′), (III′), (IIIa′), (IIIb′), (IV), (IVa), (IVa′), and (IVa″), or pharmaceutically acceptable salts thereof, R³ can comprise an ionizable group, such as groups that can be cationic depending on pH, including amine, heterocyclyl (e.g., imidazole and pyridine), urea or guanidinyl groups. In some peptidomimetic compounds, or a pharmaceutically acceptable salt thereof, the heterocyclyl group is an imidazole, triazole, pyrrole, tetrazole, and the like. In still other of the peptidomimetic compounds, or a pharmaceutically acceptable salt thereof, can be a thiophenyl group or an amide group.

In the peptidomimetic compounds of formula (I), (Ia), (II), (IIa), (III), (IIIa), (IIIb), (I′), (Ia′), (II′), (IIa′), (III′), (IIIa′), (IIIb′), (IV), (IVa), (IVa′), and (IVa″), or pharmaceutically acceptable salts thereof, R³ can be or comprise a group of the formula:

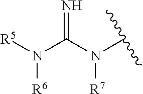

wherein: R⁵ is H or alkyl; R⁶ is H, alkyl or haloalkyl, such as fluoroalkyl; and R⁷ is H or alkyl or R⁶ and R⁷, together with the atoms to which they are attached, can join to form a five- or six-membered ring. Thus, for example, R³ can be:

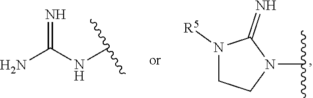

such that R³-L¹ can be or comprise groups of the formula:

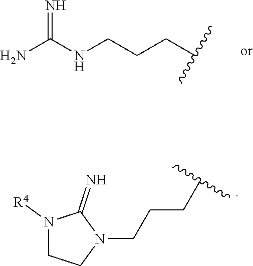

Examples of compounds of the formulae (I), (Ia), (I′), (Ia′), (IV), and (IVa′), include, but are not limited to, the compounds listed in Table 1 and pharmaceutically acceptable salts thereof:

TABLE 1

| | | UBR1 | |
|---|---|---|---|
| | | A | UBR2 |
| | | Affinit | Affinit |
| Compound | | y Ki FP | y Ki FP |
| Number | Compound structure | (μM) | (μM) |
| 1 | 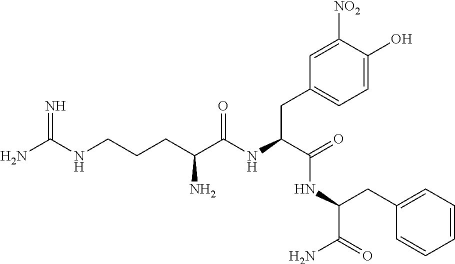 [SMILES: N=C(N)NCCC[C@H](N)C(=O)N[C@@H](Cc1ccc(O)c([N+](=O)[O-])c1)C(=O)N[C@@H](Cc1ccccc1)C(N)=O] | ++ | ++ |
| 2 | 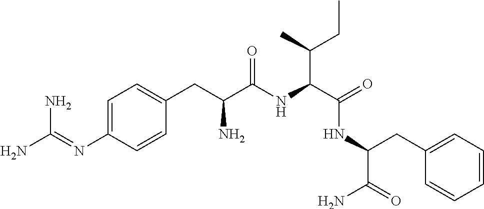 [SMILES: CC[C@H](C)[C@H](NC(=O)[C@@H](N)Cc1ccc(N=C(N)N)cc1)C(=O)N[C@@H](Cc1ccccc1)C(N)=O] | ++ | +++ |
| 3 | 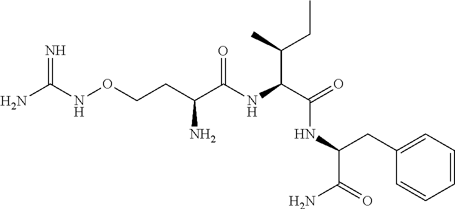 [SMILES: CC[C@H](C)[C@H](NC(=O)[C@@H](N)CCONC(=N)N)C(=O)N[C@@H](Cc1ccccc1)C(N)=O] | + | + |
| 4 | 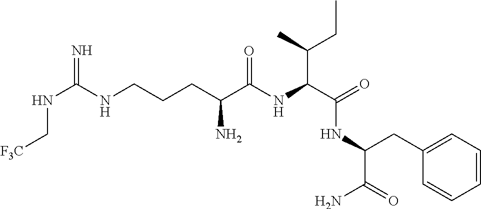 [SMILES: CC[C@H](C)[C@H](NC(=O)[C@@H](N)CCCNC(=N)NCC(F)(F)F)C(=O)N[C@@H](Cc1ccccc1)C(N)=O] | +++ | +++ |
| 5 | 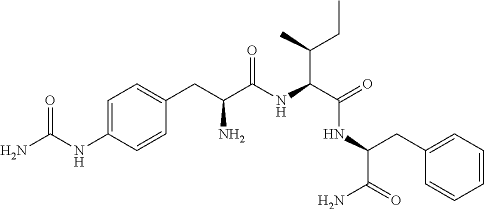 [SMILES: CC[C@H](C)[C@H](NC(=O)[C@@H](N)Cc1ccc(NC(N)=O)cc1)C(=O)N[C@@H](Cc1ccccc1)C(N)=O] | >100 | + |
| 6 | 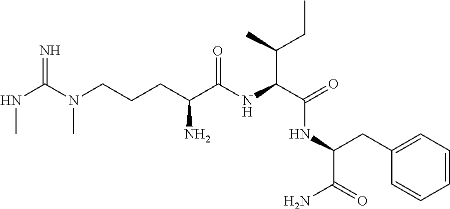 [SMILES: CC[C@H](C)[C@H](NC(=O)[C@@H](N)CCCN(C)C(=N)NC)C(=O)N[C@@H](Cc1ccccc1)C(N)=O] | >100 | >100 |
| 7 | 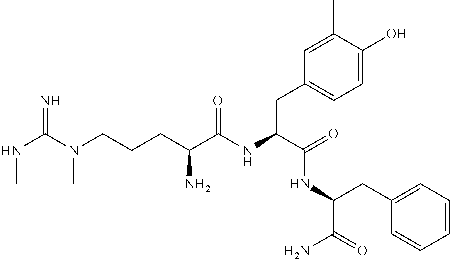 [SMILES: CNC(=N)N(C)CCC[C@H](N)C(=O)N[C@@H](Cc1ccc(O)c(C)c1)C(=O)N[C@@H](Cc1ccccc1)C(N)=O] | + | + |
| 8 | 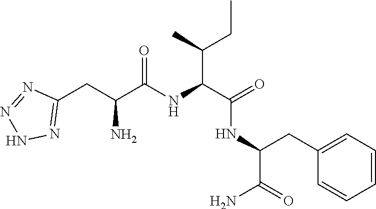 [SMILES: CC[C@H](C)[C@H](NC(=O)[C@@H](N)Cc1nn[nH]n1)C(=O)N[C@@H](Cc1ccccc1)C(N)=O] | >100 | >100 |
| 9 | 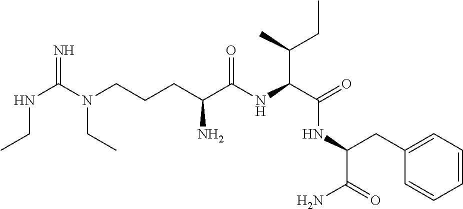 [SMILES: CCNC(=N)N(CC)CCC[C@H](N)C(=O)N[C@H](C(=O)N[C@@H](Cc1ccccc1)C(N)=O)[C@@H](C)CC] | + | + |
| 10 | 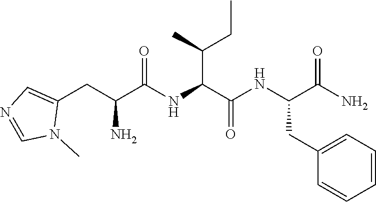 [SMILES: CC[C@H](C)[C@H](NC(=O)[C@@H](N)Cc1cncn1C)C(=O)N[C@@H](Cc1ccccc1)C(N)=O] | >100 | >100 |
| 11 | 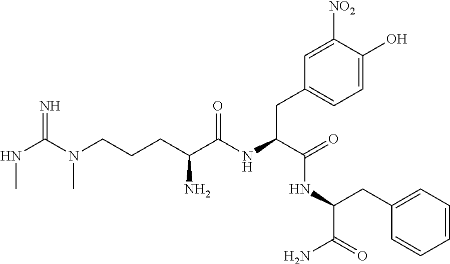 [SMILES: CNC(=N)N(C)CCC[C@H](N)C(=O)N[C@@H](Cc1ccc(O)c([N+](=O)[O-])c1)C(=O)N[C@@H](Cc1ccccc1)C(N)=O] | + | ++ |
| 12 | 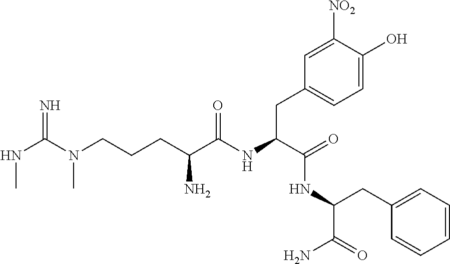 [SMILES: CNC(=N)N(C)CCC[C@H](N)C(=O)N[C@@H](Cc1ccc(O)c([N+](=O)[O-])c1)C(=O)N[C@@H](Cc1ccccc1)C(N)=O] | ++ | +++ |
| 13 | 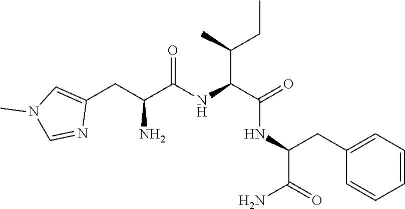 [SMILES: CC[C@H](C)[C@H](NC(=O)[C@@H](N)Cc1cn(C)cn1)C(=O)N[C@@H](Cc1ccccc1)C(N)=O] | >100 | >100 |
| 14 | 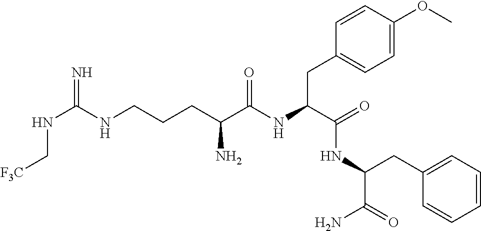 [SMILES: COc1ccc(C[C@H](NC(=O)[C@@H](N)CCCNC(=N)NCC(F)(F)F)C(=O)N[C@@H](Cc2ccccc2)C(N)=O)cc1] | +++ | +++ |
| 15 | 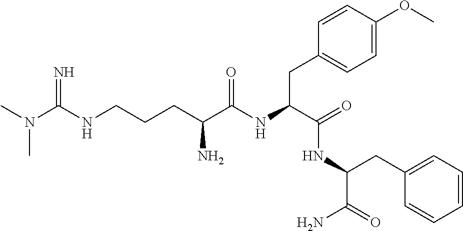 [SMILES: COc1ccc(C[C@H](NC(=O)[C@@H](N)CCCNC(=N)N(C)C)C(=O)N[C@@H](Cc2ccccc2)C(N)=O)cc1] | +++ | +++ |
| 16 | 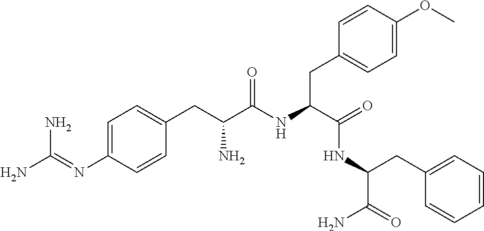 [SMILES: COc1ccc(C[C@H](NC(=O)[C@H](N)Cc2ccc(N=C(N)N)cc2)C(=O)N[C@@H](Cc2ccccc2)C(N)=O)cc1] | +++ | +++ |
| 17 | 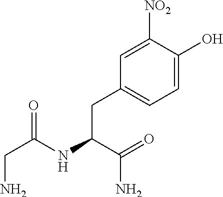 [SMILES: NCC(=O)N[C@@H](Cc1ccc(O)c([N+](=O)[O-])c1)C(N)=O] | >100 | >100 |
| 18 | 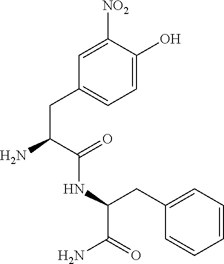 [SMILES: NC(=O)[C@H](Cc1ccccc1)NC(=O)[C@@H](N)Cc1ccc(O)c([N+](=O)[O-])c1] | >100 | >100 |
| 19 | 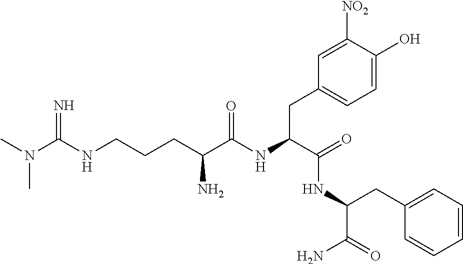 [SMILES: CN(C)C(=N)NCCC[C@H](N)C(=O)N[C@@H](Cc1ccc(O)c([N+](=O)[O-])c1)C(=O)N[C@@H](Cc1ccccc1)C(N)=O] | +++ | +++ |
| 20 | 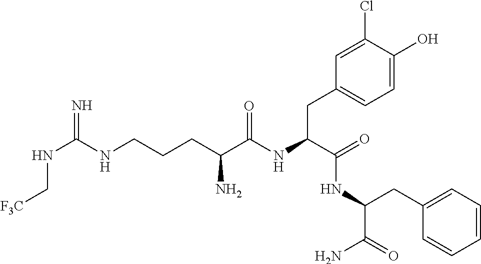 [SMILES: N=C(NCCC[C@H](N)C(=O)N[C@@H](Cc1ccc(O)c(Cl)c1)C(=O)N[C@@H](Cc1ccccc1)C(N)=O)NCC(F)(F)F] | +++ | +++ |
| 21 | 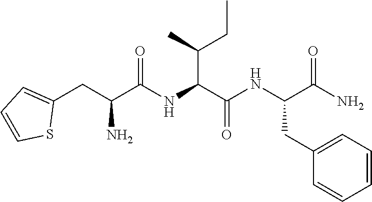 [SMILES: CC[C@H](C)[C@H](NC(=O)[C@@H](N)Cc1cccs1)C(=O)N[C@@H](Cc1ccccc1)C(N)=O] | >100 | >100 |
| 22 | 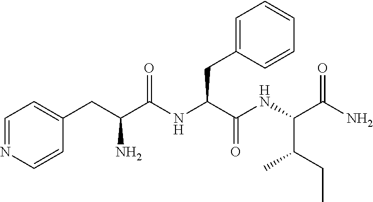 [SMILES: CC[C@H](C)[C@H](NC(=O)[C@H](Cc1ccccc1)NC(=O)[C@@H](N)Cc1ccncc1)C(N)=O] | >100 | >100 |
| 23 | 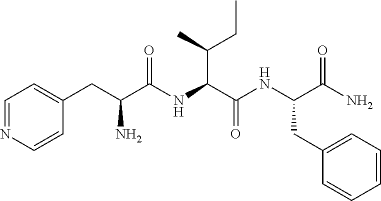 [SMILES: CC[C@H](C)[C@H](NC(=O)[C@@H](N)Cc1ccncc1)C(=O)N[C@@H](Cc1ccccc1)C(N)=O] | >100 | >100 |
| 24 | 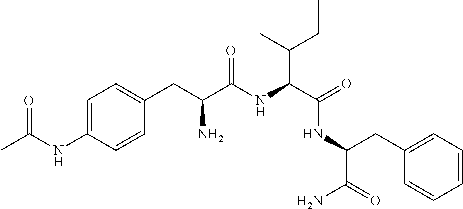 [SMILES: CCC(C)[C@H](NC(=O)[C@@H](N)Cc1ccc(NC(C)=O)cc1)C(=O)N[C@@H](Cc1ccccc1)C(N)=O] | + | + |
| 25 | 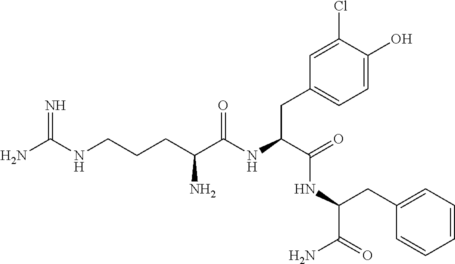 [SMILES: N=C(N)NCCC[C@H](N)C(=O)N[C@@H](Cc1ccc(O)c(Cl)c1)C(=O)N[C@@H](Cc1ccccc1)C(N)=O] | ++ | + |
| 26 | 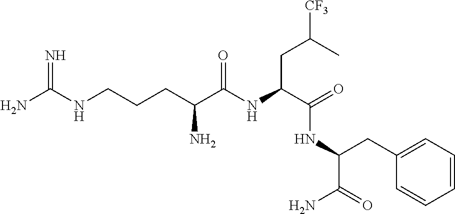 [SMILES: CC(C[C@H](NC(=O)[C@@H](N)CCCNC(=N)N)C(=O)N[C@@H](Cc1ccccc1)C(N)=O)C(F)(F)F] | +++ | +++ |
| 27 | 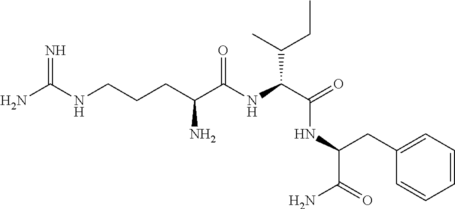 | ++ | ++ |
| 28 | 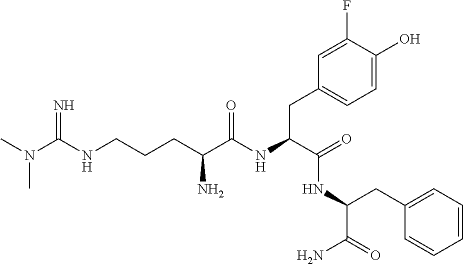 [SMILES: CN(C)C(=N)NCCC[C@H](N)C(=O)N[C@@H](Cc1ccc(O)c(F)c1)C(=O)N[C@@H](Cc1ccccc1)C(N)=O] | +++ | ++ |
| 29 | 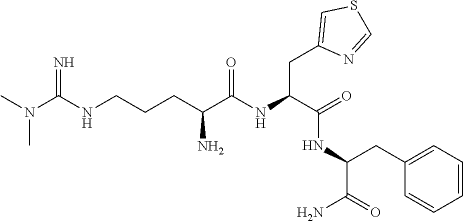 [SMILES: CN(C)C(=N)NCCC[C@H](N)C(=O)N[C@@H](Cc1cscn1)C(=O)N[C@@H](Cc1ccccc1)C(N)=O] | ++ | ++ |
| 30 | 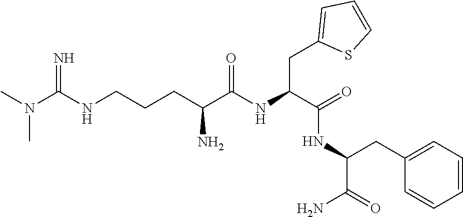 [SMILES: CN(C)C(=N)NCCC[C@H](N)C(=O)N[C@@H](Cc1cccs1)C(=O)N[C@@H](Cc1ccccc1)C(N)=O] | +++ | ++ |
| 31 | 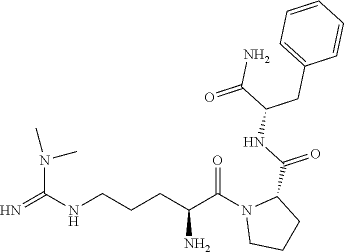 [SMILES: CN(C)C(=N)NCCC[C@H](N)C(=O)N1CCC[C@H]1C(=O)N[C@@H](Cc1ccccc1)C(N)=O] | >100 | >100 |
| 32 | 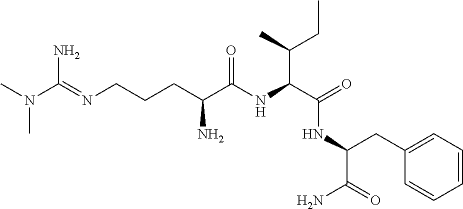 [SMILES: CC[C@H](C)[C@H](NC(=O)[C@@H](N)CCC/N=C(\N)N(C)C)C(=O)N[C@@H](Cc1ccccc1)C(N)=O] | ++ | ++ |
| 33 | 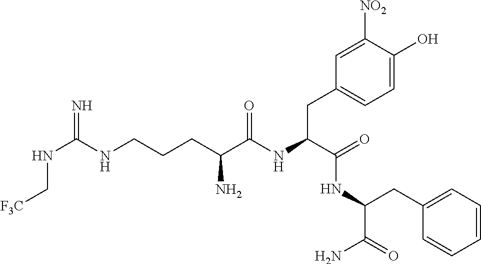 [SMILES: N=C(NCCC[C@H](N)C(=O)N[C@@H](Cc1ccc(O)c([N+](=O)[O-])c1)C(=O)N[C@@H](Cc1ccccc1)C(N)=O)NCC(F)(F)F] | +++ | +++ |

+, 100 <, >10 μM
++, <10, >1 μM
+++, <1 μM

### Pharmaceutical Compositions, Routes of Administration, and Dosing

The disclosure also provides a pharmaceutical composition comprising a compound of any of the preceding formulae and a pharmaceutically acceptable carrier. The disclosure also provides a pharmaceutical composition comprising a therapeutically effective amount of a compound of one of formulae (I), (Ia), (II), (IIa), (III), (IIIa), (IIIb), (I′), (Ia′), (II′), (IIa′), (III′), (IIIa′), (IIIb′), (IV), (IVa), (IVa′), and (IVa″) and a pharmaceutically acceptable carrier.

The disclosure also contemplates pharmaceutical compositions comprising one or more compounds of the various embodiments of the disclosure and one or more pharmaceutically acceptable excipients. A “pharmaceutical composition” refers to a chemical or biological composition suitable for administration to a subject (e.g., mammal). Such compositions can be specifically formulated for administration via one or more of a number of routes, including but not limited to buccal, cutaneous, epicutaneous, epidural, infusion, inhalation, intraarterial, intracardial, Intracerebroventricular, intraderrnal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. In addition, administration can by means of capsule, drops, foams, gel, gum, injection, liquid, patch, pill, porous pouch, powder, tablet, or other suitable means of administration.

A “pharmaceutical excipient” or a “pharmaceutically acceptable excipient” is a carrier, sometimes a liquid, in which an active therapeutic agent is formulated. The excipient generally does not provide any pharmacological activity to the formulation, though it can provide chemical and/or biological stability, and release characteristics. Examples of suitable formulations can be found, for example, in Remington, The Science And Practice of Pharmacy, 20th Edition, (Gennaro, A. R., Chief Editor), Philadelphia College of Pharmacy and Science, 2000, which is incorporated by reference in its entirety.

As used herein “phannaceutically acceptable carrier” or “excipient” includes, but is not limited to, any and all solvents, dispersion media, coatings, antibacterial and antifungal agents, isotonic and absorption delaying agents that are physiologically compatible. In one embodiment, the carrier is suitable for parenteral administration. Alternatively, the carrier can be suitable for intravenous, intraperitoneal, intramuscular, sublingual, or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the disclosure is contemplated. Supplementary active compounds can also be incorporated into the compositions.

Pharmaceutical compositions can be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (e.g., glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants.

In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, the compounds described herein can be formulated in a time release formulation, for example in a composition that includes a slow release polymer. The active compounds can be prepared with carriers that will protect the compound against rapid release, such as a controlled release formulation, including implants and microencapsulated delivery systems. Biodegradable, biocomnpatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are known to those skilled in the art.

Oral forms of administration are also contemplated herein. The pharmaceutical compositions of the disclosure can be orally administered as a capsule (hard or soft), tablet (film coated, enteric coated or uncoated), powder or granules (coated or uncoated) or liquid (solution or suspension). The formulations can be conveniently prepared by any of the methods well-known in the art. The pharmaceutical compositions of the disclosure can include one or more suitable production aids or excipients including fillers, binders, disintegrants, lubricants, diluents, flow agents, buffering agents, moistening agents, preservatives, colorants, sweeteners, flavors, and pharmaceutically compatible carriers.

For each of the recited embodiments, the compounds can be administered by a variety of dosage forms as known in the art. Any biologically-acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, gum, granules, particles, microparticles, dispersible granules, cachets, douches, suppositories, creams, topicals, inhalants, aerosol inhalants, patches, particle inhalants, implants, depot implants, ingestibles, injectables (including subcutaneous, intramuscular, intravenous, and intradernal), infusions, and combinations thereof.

Other compounds which can be included by admixture are, for example, medically inert ingredients (e.g., solid and liquid diluent), such as lactose, dextrosesaccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate and/or polyethylene glycols; gelling agents such as colloidal days; thickening agents such as gum tragacanth or sodium alginate, binding agents such as starches, arabic gums, gelatin, methylcellulose, carboxymethylcellulose or polyvinylpyrrolidone; disintegrating agents such as starch, alginic acid, alginates or sodium starch glycolate; effervescing mixtures; dyestuff; sweeteners; wetting agents such as lecithin, poiysorbates or laurylsulphates; and other therapeutically acceptable accessory ingredients, such as humectants, preservatives, buffers and antioxidants, which are known additives for such formulations.

Liquid dispersions for oral administration can be syrups, emulsions, solutions, or suspensions. The syrups can contain as a carrier, for example, saccharose or saccharose with glycerol and/or mannitol and/or sorbitol. The suspensions and the emulsions can contain a carrier, for example a natural gum, agar, sodium alginate, pectin, methylcellulose, carboxymethylcellulose, or polyvinyl alcohol.

The amount of active compound in a therapeutic composition according to various embodiments of the disclosure can vary according to factors such as the disease state, age, gender, weight, patient history, risk factors, predisposition to disease, administration route, pre-existing treatment regime (e.g., possible interactions with other medications), and weight of the subject. Dosage regimens can be adjusted to provide the optimum therapeutic response. For example, a single bolus can be administered, several divided doses can be administered over time, or the dose can be proportionally reduced or increased as indicated by the exigencies of therapeutic situation.

A “dosage unit form,” as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of sensitivity in subjects, in therapeutic use for treatment of conditions in mammals (e.g., humans) for which the compounds of the disclosure or an appropriate pharmaceutical composition thereof are effective, the compounds of the disclosure can be administered in an effective amount. The dosages as suitable for this disclosure can be a composition, a pharmaceutical composition or any other compositions described herein.

For each of the recited embodiments, the dosage is typically administered once, twice, or thrice a day, although more frequent dosing intervals are possible. The dosage can be administered every day, every 2 days, every 3 days, every 4 days, every 5 days, every 6 days, and/or every 7 days (once a week). In one embodiment, the dosage can be administered daily for up to and including 30 days, preferably between 7-10 days. In another embodiment, the dosage can be administered twice a day for 10 days. If the patient requires treatment for a chronic disease or condition, the dosage can be administered for as long as signs and/or symptoms persist. The patient can require “maintenance treatment” where the patient is receiving dosages every day for months, years, or the remainder of their lives. In addition, the composition of this disclosure can be to effect prophylaxis of recurring symptoms. For example, the dosage can be administered once or twice a day to prevent the onset of symptoms in patients at risk, especially for asymptomatic patients.

The compositions described herein can be administered in any of the following routes: buccal, epicutaneous, epidural, infusion, inhalation, intraarterial, intracardial, intracerebroventricular, intradermal, intramuscular, intranasal, intraocular, intraperitoneal, intraspinal, intrathecal, intravenous, oral, parenteral, pulmonary, rectally via an enema or suppository, subcutaneous, subdermal, sublingual, transdermal, and transmucosal. The preferred routes of administration are buccal and oral. The administration can be local, where the composition is administered directly, close to, in the locality, near, at, about, or in the vicinity of, the site(s) of disease, e.g., inflammation, or systemic, wherein the composition is given to the patient and passes through the body widely, thereby reaching the site(s) of disease. Local administration can be administration to, for example, tissue, organ, and/or organ system, which encompasses and/or is affected by the disease, and/or where the disease signs and/or symptoms are active or are likely to occur. Administration can be topical with a local effect, composition is applied directly where its action is desired. Administration can be enteral wherein the desired effect is systemic (non-local), composition is given via the digestive tract. Administration can be parenteral, where the desired effect is systemic, composition is given by other routes than the digestive tract.

As stated in the disclosure, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound of the disclosure being administered, the size of the subject, or the severity of the disease or condition. One of ordinary skill in the art can empirically determine the effective amount of a particular compound of the disclosure and/or other therapeutic agent without necessitating undue experimentation. A maximum dose may be used, that is, the highest safe dose according to some medical judgment. Multiple doses per day may be contemplated to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1000 milligrams/kg per day. Oral doses in the range of 0.5 to 50 milligrams/kg, in one or more administrations per day, can yield therapeutic results. Dosage may be adjusted appropriately to achieve desired drug levels, local or systemic, depending upon the mode of administration. For example, intravenous administration may vary from one order to several orders of magnitude lower dose per day, in the event that the response in a subject is insufficient at such doses, even higher doses (or effective higher doses by a different, more localized delivery route) may be employed to the extent that patient tolerance permits. Multiple doses per day are contemplated to achieve appropriate systemic levels of the compound.

For any compound described herein the therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

For clinical use, any compound of the disclosure can be administered In an amount equal or equivalent to 0.2-2000 milligram (mg) of compound per kilogram (kg) of body weight of the subject per day. The compounds of the disclosure can be administered in a dose equal or equivalent to 2-2000 mg of compound per kg body weight of the subject per day. The compounds of the disclosure can be administered In a dose equal or equivalent to 20-2000 mg of compound per kg body weight of the subject per day. The compounds of the disclosure can be administered in a dose equal or equivalent to 50-2000 mg of compound per kg body weight of the subject per day. The compounds of the disclosure can be administered in a dose equal or equivalent to 100-2000 mg of compound per kg body weight of the subject per day. The compounds of the disclosure can be administered in a dose equal or equivalent to 200-2000 mg of compound per kg body weight of the subject per day. Where a precursor or prodrug of the compounds of the disclosure is to be administered rather than the compound itself, it is administered in an amount that is equivalent to, i.e., sufficient to deliver, the above-stated amounts of the compounds of the technology.

The formulations of the compounds of the disclosure can be administered to human subjects in therapeutically effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments may be used to optimize the dose and dosing frequency for any particular compound.

The compounds of the disclosure can be administered at a concentration in the range from about 0.001 microgram/kg to greater than about 500 mg/kg. For example, the concentration may be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof, it is to be understood that all values and ranges between these values and ranges are meant to be encompassed by the present technology.

The compounds of the disclosure can be administered at a dosage in the range from about 0.2 milligram/kg/day to greater than about 100 mg/kg/day. For example, the dosage may be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compounds of the disclosure can be administered at a dosage in the range from about 0.25 milligram/kg/day to about 25 mg/kg/day. For example, the dosage may be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/lay, 45 mg/kg/day, 46 mg/kg/day, 47 mg/kg/day, 48 mg/kg/day, 49 mg/kg/day, or 50 mg/kg/day.

In various embodiments, the compound or precursor thereof is administered in concentrations that range from 0.01 micromolar to greater than or equal to 500 micromolar. For example, the dose may be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof, it is to be understood that all values and ranges between these values and ranges are meant to be encompassed by the present technology.

In various embodiments, the compound or precursor thereof is administered at concentrations that range from 0.10 microgram/mL to 500.0 microgram/mL. For example, the concentration may be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL, 30 microgram/mL, 35 microgram/mL, 40 microgram/mL, 45 microgram/mL, 50 microgram/mL, 60.0 micromolar/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 micromolar/mL, 400.0 microgram/mL, 450.0 microgram/mL, to greater than about 500.0 microgram/mL or any incremental value thereof. It is to be understood that all values and ranges between these values and ranges are meant to be encompassed by the present Technology.

The disclosure also provides a method for degrading a target substrate protein comprising administering a therapeutically effective amount of any of the preceding compounds, e g., a compound of any of formulae (I), (Ia), (II), (IIa), (III), (IIa), (I′), (Ia′), (II′), (IIa′), (III′), (IIIa′), (IV), (IVa), (IVa′), and (IVa″), or a pharmaceutical composition comprising said compound, to a subject in need thereof.

The disclosure also provides a method for treating cancer and other proliferative disorders, including, but not limited to breast cancer, cervical cancer, colon and rectal cancer, leukemia, lung cancer (e.g., non-small cell lung cancer), melanoma, multiple myeloma, non-Hodgkin's lymphoma, ovarian cancer, pancreatic cancer, prostate cancer, gastric cancer, leukemias (e.g., myeloid, lymphocytic, myelocytic and lymphoblastic leukemias), malignant melanomas, and T-cell lymphoma) or neuroblastoma comprising administering a therapeutically effective amount of any of the preceding compounds, e.g., a compound of any of formulae (I), (Ia), (II), (IIa), (III), (IIIa), (IIIb), (I′), (Ia′), (II′), (IIa′), (III′), (IIIa′), (IIIb′), (IV), (IVa), (IVa′), and (IVa″), or a pharmaceutical composition comprising said compound, to a subject in need thereof

### Kits

In another embodiment, the kit or container can hold a therapeutically effective amount of a pharmaceutical composition for treating, preventing, or controlling a disease and instructions for using the pharmaceutical composition for control of the disease. The pharmaceutical composition can include the peptidomimetic compounds described herein in a therapeutically effective amount such that the disease is controlled, prevented, or treated. Such a composition can be in liquid form, powder form or other form permitting ready administration to a patient.

Kits can also comprise containers with tools useful for administering the compositions of the invention. Such tools can include syringes, swabs, catheters, antiseptic solutions, and the like. Some kits can include all of the desired tools, solutions, compounds, including mixing vessels, utensils, and injection devices, to treat a patient according to any of the methods described herein. In one embodiment, a kit includes peptidomimetics compounds of the various embodiments described herein. The peptidomimetics compounds can be sterile-packaged as a dry powder in a suitable container (e.g., a substantially water-impermeable) such as a syringe, vial (e.g., the vial can include a septum and/or a crimp seal; and the vial can optionally comprise an inert atmosphere, such as a nitrogen atmosphere or dry air) or pouch (e.g., a pouch comprising a moisture barrier; and the pouch can optionally comprise an inert atmosphere, such as a nitrogen atmosphere, or dry air). The vial containing peptidomimetics compounds can have an injection cap that does not require the use of a needle to withdraw the suspended solution can be used to avoid damaging the peptidomimetics compounds or separating the particles from the solution under negative pressure. The kit can also include a desiccant. The desiccant can be included in the pouch or integrated into the layers of the pouch material. In some embodiments, the peptidomimetics compounds can be sterile-packaged in frozen vehicle. As mentioned previously, the vehicle can be any suitable vehicle, including flowable vehicles (e.g., a liquid vehicle) such as a flowable, bioresorbable polymer, saline, sterile water, Ringer's solutions, and isotonic sodium chloride solutions. Examples of vehicles include, but are not limited, to Sodium Chloride Injection USP (0.9%), Ringer's Injection USP, Lactated Ringer's Injection USP, Sodium Lactate Injection USP, Dextrose Injection USP (5% or 10%), Bacteriostatic Water for Injection USP and Sterile Water for Injection USP. In some examples, the peptidomimetics compounds can be suspended in water; pre-filled into a container, such as a syringe; and frozen.

The kit can include at least one static mixing element, such as a one that is attached to a syringe. In some embodiments, the user provides a static mixing element to deliver the peptidomimetics compounds.

The kit can also include beads that serve to, among other things, disaggregate any agglomeration of the peptidomimetics compounds that can occur when the peptidomimetics compounds of the various embodiments described herein are reconstituted with a vehicle. In some embodiments, the beads are sufficiently larger than the peptidomimetics compounds, so that the peptidomimetics compounds can be selectively delivered to the injection site, while the beads remain in the injection device (e.g., a syringe). For example, the beads can have at least one dimension that is about 1 mm. The beads can be of any suitable shape, including spherical and oval in shape. The beads can also have any suitable texture. For example, the beads can have a smooth texture and/or a rough texture. The beads can also be made of any suitable material, including glass, ceramic, metal (e.g. stainless steel), polymeric (e.g. ePTFE or polypropylene), and composite materials. The beads can be included in the kit in a separate container; in the same container as the peptidomimetics compounds of the various embodiments described herein; or the user can provide beads of suitable size, shape, texture, and/or materials at the point of care.

The kit can also include an injection vehicle described herein, such as sterile water or sterile saline (e.g., in the case where the target injection area is substantially hydrophobic or lipophilic) or other suitable vehicle, including a non-aqueous vehicle (e.g., a hydrophobic, liquid vehicle described herein). Prior to administration, the peptidomimetics compounds can be added to the injection vehicle to form a suspension and agitated (e.g., stirred, shaken or vortexed) to maximize homogeneity. In some embodiments, the peptidomimetics compounds can come in the kit, suspended in a vehicle, such as a non-aqueous vehicle (e.g., a hydrophobic, liquid vehicle described herein).

The kit can further include a hypodermic needle or other delivery device, such as a cannula, catheter, or other suitable tubing. The kit can further include instructions, dosage tables, and other pertinent information for a practitioner.

The kit can include one or more additional Active Pharmaceutical Ingredients (APIs), such as an antiemetic or an antidiarrheal such as loperamide (Imodium®), either in the same container as the peptidomimetics compounds of the various embodiments described herein or in a separate container, such that the API in a separate container can be combined with the peptidomimetics compounds. In other embodiments, the user can provide one or more additional APIs that can be combined with the peptidomimetics compounds of the various embodiments described herein, at the point of care.

The kits can include instructions or printed indicia, to provide for directions for reconstituting the contents of the multiple packages, and/or for the administration of the resulting composition (e.g., the injectable compositions). For example, instructions on printed indicia can instruct injection into biological tissue including at least one of fatty tissue, epidural tissue, and at or near a targeted nerve.

### Definitions

The term “therapeutically effective amount” as used herein, refers to that amount of one or more compounds of the various examples of the disclosure that elicits a biological or medicinal response in a tissue system, animal or human, that is being sought by a researcher, veterinarian, medical doctor or other clinician, which includes alleviation of the symptoms of the disease or disorder being treated. In some examples, the therapeutically effective amount is that which can treat or alleviate the disease or symptoms of the disease at a reasonable benefit/risk ratio applicable to any medical treatment. However, it is to be understood that the total daily usage of the compounds and compositions described herein can be decided by the attending physician within the scope of sound medical judgment. The specific therapeutically-effective dose level for any particular patient will depend upon a variety of factors, including the condition being treated and the severity of the condition; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician. It is also appreciated that the therapeutically effective amount can be selected with reference to any toxicity, or other undesirable side effect, that might occur during administration of one or more of the compounds described herein.

The term “alkyl” as used herein refers to substituted or unsubstituted straight chain, branched and cyclic, saturated mono- or bi-valent groups having from 1 to 20 carbon atoms, 10 to 20 carbon atoms, 12 to 18 carbon atoms, 6 to about 10 carbon atoms, 1 to 10 carbons atoms, 1 to 8 carbon atoms, 2 to 8 carbon atoms, 3 to 8 carbon atoms, 4 to 8 carbon atoms, 5 to 8 carbon atoms, 1 to 6 carbon atoms, 2 to 8 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂—, and —CH₂CH₂CH₂CH₂CH₂—. Examples of branched bi-valent alkyl groups include —CH(CH₃)CH₂— and —CH₂CH(CH₃)CH₂—. Examples of cyclic alkyl groups include cyclopropyl, cyclobutyl, cyclopently, cyclohexyl, cyclooctyl, bicyclo[1.1.1]pentyl, bicyclo[2.1.1]hexyl, and bicyclo[2.2.1]heptyl. Cycloalkyl groups further include polycyclic cycloalkyl groups such as, but not limited to, norbornyl, adamantyl, bornyl, camphenyl, isocamphenyl, and carenyl groups, and fused rings such as, but not limited to, docalinyl, and the like. In some embodiments, alkyl includes a combination of substituted and unsubstituted alkyl. As an example, alkyl, and also (C₁)alkyl, includes methyl and substituted methyl. As a particular example, (C₁)alkyl includes benzyl As a further example, alkyl can include methyl and substituted (C₂-C₈)alkyl. Alkyl can also include substituted methyl and unsubstituted (C₂-C₈)alkyl. In some embodiments, alkyl can be methyl and C₂-C₈ linear alkyl. In some embodiments, alkyl can be methyl and C₂-C₈ branched alkyl. The term methyl is understood to be —CH₃, which is not substituted. The term methylene is understood to be —CH₂—, which is not substituted. For comparison, the term (C₁)alkyl is understood to be a substituted or an unsubstituted —CH₃ or a substituted or an unsubstituted —CH₂—. Representative substituted alkyl groups can be substituted one or more times with any of the groups listed herein, for example, cycloalkyl, heterocyclyl, aryl, amino, haloalkyl, hydroxy, cyano, carboxy, nitro, thio, alkoxy, and halogen groups. As further example, representative substituted alkyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkyl groups can be substituted from a set of groups including amino, hydroxy, cyano, carboxy, nitro, thio and alkoxy, but not including halogen groups. Thus, in some embodiments alkyl can be substituted with a non-halogen group. For example, representative substituted alkyl groups can be substituted with a fluoro group, substituted with a bromo group, substituted with a halogen other than bromo, or substituted with a halogen other than fluoro. In some embodiments, representative substituted alkyl groups can be substituted with one, two, three or more fluoro groups or they can be substituted with one, two, three or more non-fluoro groups. For example, alkyl can be trifluoromethyl, difluoromethyl, or fluoromethyl, or alkyl can be substituted alkyl other than trifluoromethyl, difluoromethyl or fluoromethyl. Alkyl can be haloalkyl or alkyl can be substituted alkyl other than haloalkyl.

The term “alkenyl” as used herein refers to substituted or unsubstituted straight chain, branched and cyclic, saturated mono- or bi-valent groups having at least one carbon-carbon double bond and from 2 to 20 carbon atoms, 10 to 20 carbon atoms, 12 to 18 carbon atoms, 6 to about 10 carbon atoms, 2 to 10 carbons atoms, 2 to S carbon atoms, 3 to S carbon atoms, 4 to S carbon atoms, 5 to S carbon atoms, 2 to 6 carbon atoms, 3 to 6 carbon atoms, 4 to 6 carbon atoms, 2 to 4 carbon atoms, or 2 to 3 carbon atoms. The double bonds can be be trans or cis orientation. The double bonds can be terminal or internal. The alkenyl group can be attached via the portion of the alkenyl group containing the double bond, e.g., vinyl, propen-1-yl and buten-1-yl, or the alkenyl group can be attached via a portion of the alkenyl group that does not contain the double bond, e.g., penten-4-yl. Examples of mono-valent (C₂-C₂₀)-alkenyl groups include those with from 1 to 8 carbon atoms such as vinyl, propenyl, propen-1-yl, propen-2-yl, butenyl, buten-1-yl, buten-2-yl, sec-buten-1-yl, sec-buten-3-yl, pentenyl, hexenyl, heptenyl and octenyl groups. Examples of branched mono-valent (C₂-C₂₀)-alkenyl groups include isopropenyl, iso-butenyl, sec-butenyl, t-butenyl, neopentenyl, and isopentenyl. Examples of straight chain bi-valent (C₂-C₂₀)alkenyl groups include those with from 2 to 6 carbon atoms such as —CH═CH—, —CH═CHCH₂—, —CH═CHCH₂CH₂—, and —CHCHCH₂CH₂CH₂—. Examples of branched bi-valent alkyl groups include —C(CH₃)=CH— and —CH=C(CH₃)CH₂—. Examples of cyclic alkenyl groups include cyclopentenyl, cyclohexenyl and cyclooctenyl. It is envisaged that alkenyl can also include masked alkenyl groups, precursors of alkenyl groups or other related groups. As such, where alkenyl groups are described it, compounds are also envisaged where a carbon-carbon double bond of an alkenyl is replaced by an epoxide or aziridine ring. Substituted alkenyl also includes alkenyl groups which are substantially tautomeric with a non-alkenyl group. For example, substituted alkenyl can be 2-aminoalkenyl, 2-alkylaminoalkenyl, 2-hydroxyalkenyl, 2-hydroxyvinyl, 2-hydroxypropenyl, but substituted alkenyl is also understood to include the group of substituted alkenyl groups other than alkenyl which are tautomeric with non-alkenyl containing groups. In some embodiments, alkenyl can be understood to include a combination of substituted and unsubstituted alkenyl. For example, alkenyl can be vinyl and substituted vinyl. For example, alkenyl can be vinyl and substituted (C₃-C₈)alkenyl. Alkenyl can also include substituted vinyl and unsubstituted (C₃-C₈)alkenyl.

Representative substituted alkenyl groups can be substituted one or more times with any of the groups listed herein, for example, monoalkylamino, dialkylamino, cyano, acetyl, amido, carboxy, nitro, alkylthio, alkoxy, and halogen groups. As further example, representative substituted alkenyl groups can be substituted one or more fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. In some embodiments, representative substituted alkenyl groups can be substituted from a set of groups including monoalkylamino, dialkylamino, cyano, acetyl, amido, carboxy, nitro, alkylthio and alkoxy, but not including halogen groups. Thus, in some embodiments alkenyl can be substituted with a non-halogen group. In some embodiments, representative substituted alkenyl groups can be substituted with a fluoro group, substituted with a bromo group, substituted with a halogen other than bromo, or substituted with a halogen other than fluoro. For example, alkenyl can be 1-fluorovinyl, 2-fluorovinyl, 1,2-difluorovinyl, 1,2,2-trifluorovinyl, 2,2-difluorovinyl, trifluoropropen-2-yl, 3,3,3-trifluoropropenyl, 1-fluoropropenyl, 1-chlorovinyl, 2-chlorovinyl, 1,2-dichlorovinyl, 1,2,2-trichlorovinyl or 2,2-dichlorovinyl. In some embodiments, representative substituted alkenyl groups can be substituted with one, two, three or more fluoro groups or they can be substituted with one, two, three or more non-fluoro groups.

The term “alkynyl” as used herein, refers to substituted or unsubstituted straight and branched chain alkyl groups, except that at least one triple bond exists between two carbon atoms. Thus, alkynyl groups have from 2 to 50 carbon atoms, 2 to 20 carbon atoms, 10 to 20 carbon atoms, 12 to 18 carbon atoms, 6 to about 10 carbon atoms, 2 to 10 carbons atoms, 2 to 8 carbon atoms, 3 to 8 carbon atoms, 4 to 8 carbon atoms, 5 to 8 carbon atoms, 2 to 6 carbon atoms, 3 to 6 carbon atoms, 4 to 8 carbon atoms, 2 to 4 carbon atoms, or 2 to 3 carbon atoms. Examples include, but are not limited to ethynyl, propynyl, propyn-1-yl, propyn-2-yl, butynyl, butyn-1-yl, butyn-2-yl, butyn-3-yl, butyn-4-yl, pentynyl, pentyn-1-yl, hexynyl, Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “aryl” as used herein refers to substituted or unsubstituted univalent groups that are derived by removing a hydrogen atom from an arene, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is dear that the term (C₆-C₂₀)aryl encompasses mono- and polycyclic (C₆-C₂₀)aryl groups, including fused and non-fused polycyclic (C₆-C₂₀)aryl groups.

The term “heterocyclyl” as used herein refers to substituted aromatic, unsubstituted aromatic, substituted non-aromatic, and unsubstituted non-aromatic rings containing 3 or more atoms in the ring, of which, one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈). A heterocyclyl group designated as a C₂-heterocyclyl can be a 5-membered ring with two carbon atoms and three heteroatoms, a 6-membered ring with two carbon atoms and four heteroatoms and so forth. Likewise a C₄-heterocyclyl can be a 5-membered ring with one heteroatom, a 6-membered ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. Representative heterocyclyl groups include, but are not limited to piperidynyl, piperazinyl, morpholinyl, furanyl, pyrrolidinyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups. For example, heterocyclyl groups include, without limitation:

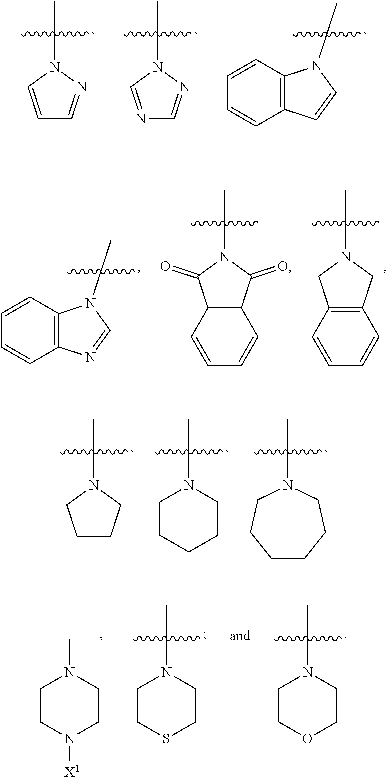

wherein X¹ represents H, (C₁-C₂₀)alkyl, (C₆-C₂₀)aryl or an amine protecting group (e.g., a t-butyloxycarbonyl group) and wherein the heterocyclyl group can be substituted or unsubstituted. A nitrogen-containing heterocyclyl group is a heterocyclyl group containing a nitrogen atom as an atom in the ring. In some embodiments, the heterocyclyl is other than thiophene or substituted thiophene. In some embodiments, the heterocyclyl is other than furan or substituted furan.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include one to about 12-20 or about 12-40 carbon atoms bonded to the oxygen atom, and can further include double or triple bonds, and can also include heteroatoms. Thus, alkyoxy also includes an oxygen atom connected to an alkyenyl group and oxygen atom connected to an alkynyl group. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “aryloxy” as used herein refers to an oxygen atom connected to an aryl group as are defined herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl, biphenylmethyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The terms “halo,” “halogen,” or “halide” group, as used herein, by themselves or as part of another substituent, mean, unless otherwise stated, a fluorine, chlorine, bromine, or iodine atom.

The term “amino” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group,

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, aryl, cycloalkyl, heterocyclyl, group or the like. The term “formyl” as used herein refers to a group containing a carbonyl moiety wherein the group is bonded via the carbonyl carbon atom. The carbonyl carbon atom is also bonded to a hydrogen atom.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom Is also bonded to an oxygen atom which is further bonded to an alkyenyl group. Alkoxycarbonyl also includes the group where a carbonyl carbon atom is also bonded to an oxygen atom which is further bonded to an alkynyl group. In a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “arylcarbonyl” as used herein refers to a group containing a carbonyl moiety wherein the group is bonded via the carbonyl carbon atom. The carbonyl carbon atom is also bonded to an aryl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to an nitrogen atom which is bonded to one or more aryl group instead of, or in addition to, the one or more alkyl group, in a further case, which is also an alkylamido as the term is defined herein, the carbonyl carbon atom is bonded to an nitrogen atom which is bonded to one or more alkenyl group instead of, or in addition to, the one or more alkyl and or/aryl group. In a further case, which is also an alkylamido as the term is defined herein, the carbonyl carbon atom is bonded to an nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “alkylthio” as used herein refers to a sulfur atom connected to an alkyl, alkenyl, or alkynyl group as defined herein.

The term “arylthio” as used herein refers to a sulfur atom connected to an aryl group as defined herein.

The term “alkylsulfonyl” as used herein refers to a sulfonyl group connected to an alkyl, alkenyl, or alkynyl group as defined herein.

The term “alkylsulfinyl” as used herein refers to a sulfinyl group connected to an alkyl, alkenyl, or alkynyl group as defined herein.

The term “dialkylaminosulfonyl” as used herein refers to a sulfonyl group connected to a nitrogen further connected to two alkyl groups, as defined herein, and which can optionally be linked together to form a ring with the nitrogen. This term also includes the group where the nitrogen is further connected to one or two alkenyl groups In place of the alkyl groups.

The term “dialkylamino” as used herein refers to an amino group connected to two alkyl groups, as defined herein, and which can optionally be linked together to form a ring with the nitrogen. This term also includes the group where the nitrogen is further connected to one or two alkenyl groups in place of the alkyl groups.

The term “dialkylamido” as used herein refers to an amide group connected to two alkyl groups, as defined herein, and which can optionally be linked together to form a ring with the nitrogen. This term also includes the group where the nitrogen is further connected to one or two alkenyl groups in place of the alkyl groups.

The term “substituted” as used herein refers to a group that is substituted with one or more groups including, but not limited to, the following groups: halogen (e.g., F, Cl, Br, and I), R, OR, OC(O)N(R)₂, CN, NO, NO₂, ONO₂, azido, CF₃, OCF₃, methylenedioxy, ethylenedioxy, (C₃-C₂₀) heteroaryl, N(R)₂, Si(R)₃, SR, SOR, SO₂R, SO₂N(R)₂, SO₂R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl or (C₆-C₂₀)aryl. Substituted also includes a group that is substituted with one or more groups including, but not limited to, the following groups: fluoro, chloro, bromo, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be Independent. For example, a substituted aryl can be substituted with bromo and a substituted heterocycle on the same compound can be substituted with alkyl. It is envisaged that a substituted group can be substituted with one or more non-fluoro groups. As another example, a substituted group can be substituted with one or more non-cyano groups. As another example, a substituted group can be substituted with one or more groups other than haloalkyl. As yet another example, a substituted group can be substituted with one or more groups other than tert-butyl. As yet a further example, a substituted group can be substituted with one or more groups other than trifluoromethyl. As yet even further examples, a substituted group can be substituted with one or more groups other than nitro, other than methyl, other than methoxymethyl, other than dialkylaminosulfonyl, other than bromo, other than chloro, other than amido, other than halo, other than benzodioxepinyl, other than polycyclic heterocyclyl, other than polycyclic substituted aryl, other than methoxycarbonyl, other than alkoxycarbonyl, other than thiophenyl, or other than nitrophenyl, or groups meeting a combination of such descriptions. Further, substituted is also understood to include fluoro, cyano, haloalkyl, tert-butyl, trifluoromethyl, nitro, methyl, methoxymethyl, dialkylaminosulfonyl, bromo, chloro, amido, halo, benzodioxepinyl, polycyclic heterocyclyl, polycyclic substituted aryl, methoxycarbonyl, alkoxycarbonyl, thiophenyl, and nitrophenyl groups.

In some instances, the compounds described herein (e.g., the compounds of the Formulae (I)-(V) can contain chiral centers. All diastereomers of the compounds described herein are contemplated herein, as well as racemates.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric (or larger) amount of the appropriate base or acid In water or In an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent Intermolecular torces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the disclosure. Examples of prodrugs include, but are not limited to, derivatives and metabolites of a compound of the disclosure that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

As used herein, the term “subject” or “patient” refers to any organism to which a composition described herein can be administered, e.g., for experimental, diagnostic, prophylactic and/or therapeutic purposes. Subject refers to a mammal receiving the compositions disclosed herein or subject to disclosed methods. It is understood and herein contemplated that “mammal” includes but is not limited to humans, non-human primates, cows, horses, dogs, cats, mice, rats, rabbits, and guinea pigs.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the disclosure, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

Each embodiment described above is envisaged to be applicable in each combination with other embodiments described herein. For example, embodiments corresponding to formula (I) are equally envisaged as being applicable to formula (Ib).

The invention will be further described by the following non-limiting examples.

### EXAMPLES

### Example 1: Expression of Ubr-Boxes as GST Fusion Proteins

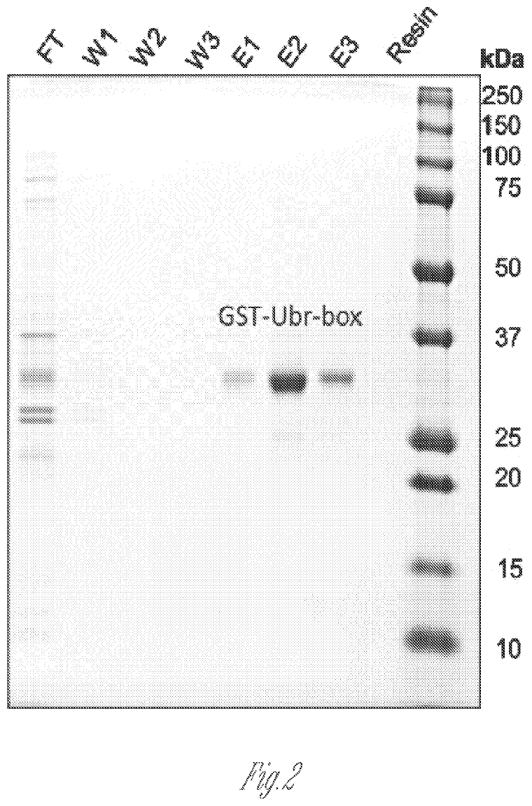

The Ubr family of E3 ligases include a 70-residue zinc finger domain called the Ubr-box that directly binds N-terminal degradation signals in substrate proteins. The Ubr-boxes of Ubr1, Ubr2, Ubr4, and Ubr5 were expressed as glutathione S-transferase fusion proteins (GST-Ubr-box), in which GST can be removed by PreScission Protease cleavage (FIG. 2). GST protein is an affinity tag that enables purification of proteins attached thereto. Glutathione is a tripeptide (Glu-Cys-Gly) that is the specific substrate for glutathione S-transferase (GST). When reduced glutathione is immobilized through its sulfhydryl group to a solid support, such as cross-linked beaded agarose, it can be used to capture pure GST or GST-tagged proteins via the enzyme-substrate binding reaction. After washing an affinity column to remove non-bound sample components, the purified GST-Ubr box protein can be dissociated and recovered (eluted) from a glutathione column by addition of excess reduced glutathione. The free glutathione competitively displaces the immobilized glutathione binding interaction with GST, allowing the GST-Ubr box protein to emerge from the affinity column.

An image of a sodium dodecyl sulfate-polyacrylamide gel electrophoresis (SDS-PAGE) gel is shown with fractions from purification of a GST-fusion Ubr-Box. Flow through (FT), wash fractions W1-W3, and elusion fractions E1-E3 are shown. Elution is complete as shown by the lack of proteins left on resin.

The GST-UBR1 fusion protein is provided as the amino acid sequence of SEQ ID NO. 3:

MSPILGYWKI KGLVQPTRLL LEYLEEKYEE HLYERDEGDK
WRNKKFELGL EFPNLPYYID GDVKLTQSMA IIRYIADKHN
MLGGCPKERA EISMLEGAVL DIRYGVSRIA YSKDFETLKV
DFLSKLPEML KMFEDRICHK TYLNGDHVTH PDFMLYDALD
VVLYMDPMCL DAFPKLVCFK KRIEAIPQID KYLKSSKYIA
WPLQGWQATF GGGDHPPKSD LEVLFQGPLG SQLCGRVFKS
GETTYSCRDC AIDPTCVLCM DCFQDSVHKN HRYKMHTSTG
GGFCDCGDTE AWKTGPFCVN HEP

The GST-UBR2 fusion protein is provided as the amino acid sequence of SEQ ID NO. 4:

MSPILGYWKI KGLVQPTRLL LEYLEEKYEE HLYERDEGDK
WRNKKFELGL EFPNLPYYID GDVKLTQSMA IIRYIADKHN
MLGGCPKERA EISMLEGAVL DIRYGVSRIA YSKDFETLKV
DFLSKLPEML KMFEDRLCHK TYLNGDHVTH PDFMLYDALD
VVLYMDPMCL DAFPKLVCFK KRIEAIPQID KYLKSSKYIA
WPLQGWQATF GGGDHPPKSD LEVLFQGPLG SLCGRVFKVG
EPTYSCRDCA VDPTCVLCME CFLGSIHRDH RYRMTTSGGG
GFCDCGDTEA WKEGPYCQKH

### Example 2: Synthesis of Tripeptide Peptidomimetic Compounds

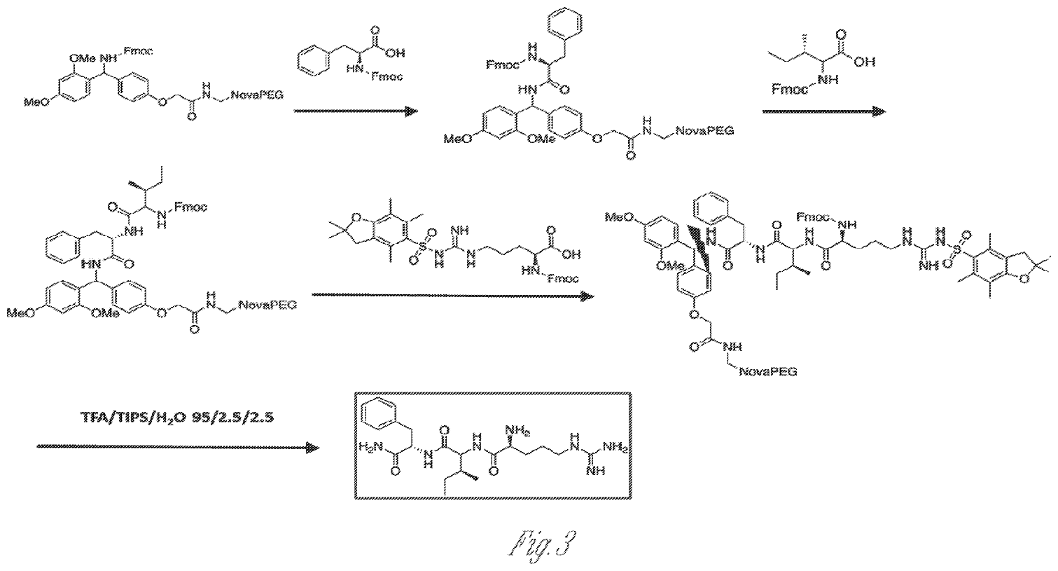

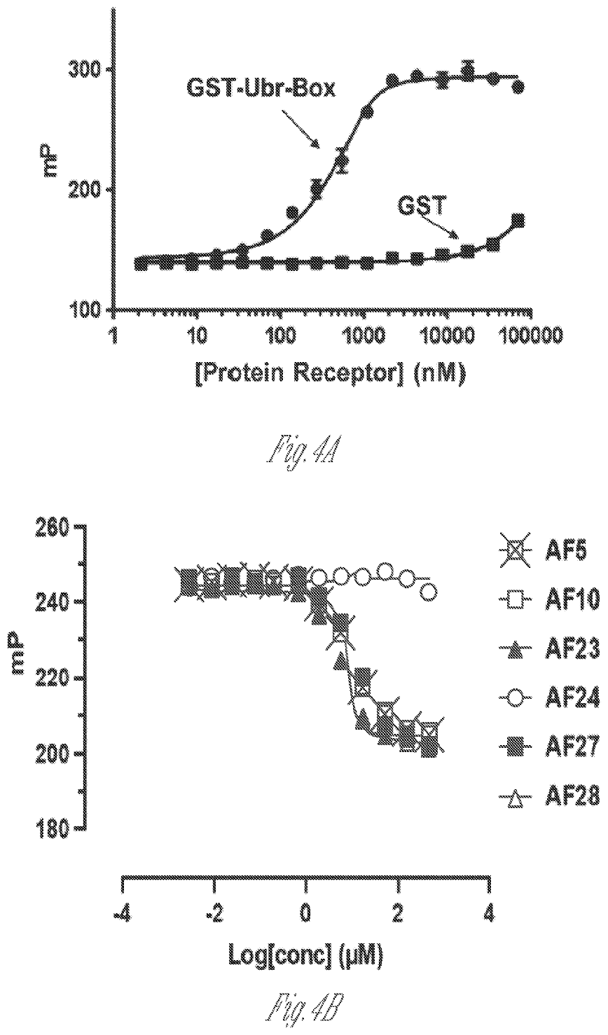

Peptidomimetic compounds that include tripeptides were synthesized by solid-phase peptide synthesis (SPPS) (FIG. 3). Solid-phase peptide synthesis reactions were conducted at molar concentrations of all reactants as following: 0.115 mmol scale for Novabiochem Resin (217 mg), fmoc protected amino acid (5 eq), HBTU/or HATU (5 eq), DIPEA (10 eq) in dry DMF. Fmoc protected amino acid was dissolved in dry DMF (3 mL) at room temperature. HATU or HBTU, and DIPEA were added to the solution and stirred for 10 min at room temperature. Next, the reaction mixture was added to the resin (preswollen for 30 min in dry DCM/DMF) and was stirred at room temperature for 1 hour 30 minutes. The reaction mixture resin was then allowed to settle down at room temperature, and the resin was washed 3 times with dry DMF and dry DCM. Each coupling was repeated twice before adding the next amino acid. Fmoc removal was performed using a solution of piperidine in EtOH/NMP, with 2*3 mL during 10 min. The resin was then dried under vacuum and cleavage from the resin was performed using a solution of TFA 19 mL+0.5 mL TIPS and 0.5 mL of water (as scavengers) during 3 hours at room temperature. The resin was washed with neat TFA, filtered and the organic phase was concentrated under reduced pressure. The residue was redissolved in ACN/Water (1 mL), lyophilized overnight and was purified on HPLC. The product fraction from the column, upon the mass spectrometry of the fraction, was collected, solvent was removed, and the content was lyophilized to get the desired products.

### Example 3: Determination of Peptidomimetic Compounds' Binding Affinity for Ubr-Box

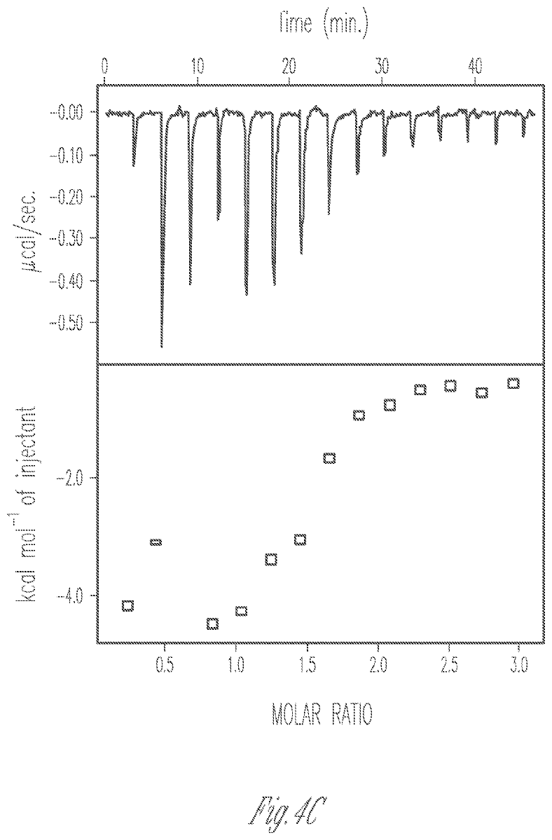

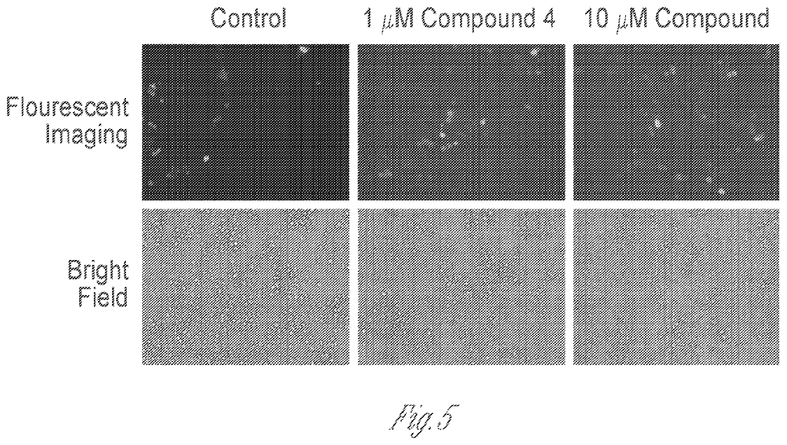

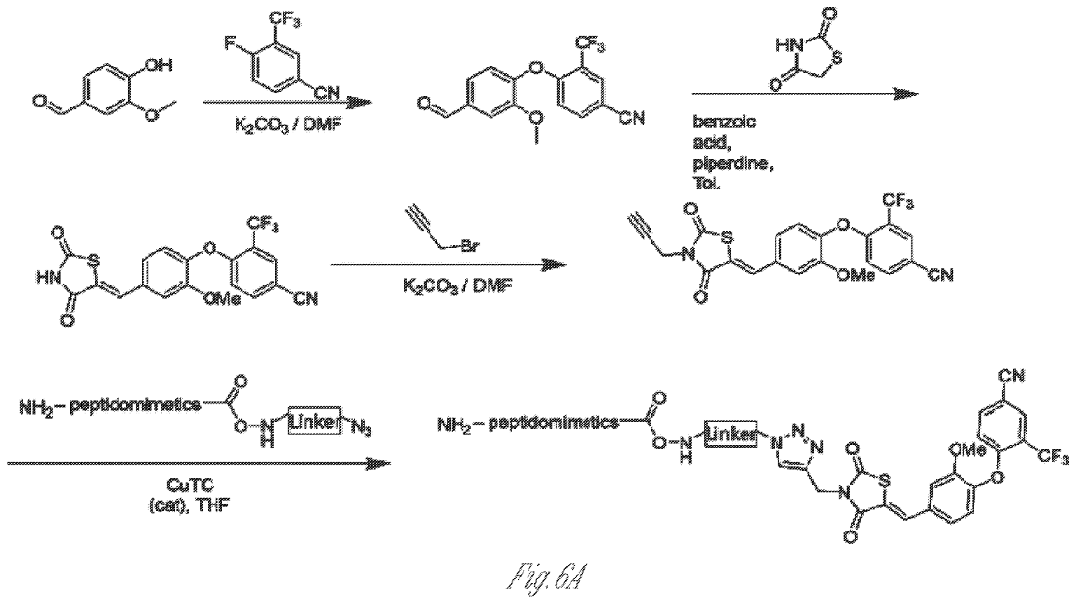

Fluorescence Polarization (FP) assays were used to determine binding affinity of the peptidomimetic compounds for the Ubr box (FIGS. 4A-B). FIG. 1A depicts the mechanism of action of peptidomimetic compounds that include a linker. FIG. 1B depicts the mechanism of action of peptidomimetic compounds that do not include a linker. Binding affinities as determined by Structure Activity Relationship Studies (SARs) of additional peptidomimetic compounds to GST-Ubr-box of Ubr 1 and Ubr2 are shown in Table 1.

The FP assay uses a tetrapeptide with a sequence arginine-isoleucine-phenylalanine-serine (RIFS) with fluorescein attached to the C-terminus as tracer that was previously shown to bind to the Ubr-boxes of Ubr1 and Ubr2 with similar binding affinity. Assays were developed in 384-well plates, which were read on a Clariostar Reader. Signal-to-background ratio (S/B) was optimized by varying concentrations of GST-Ubr-box and tracer concentrations. GST-fusion proteins were used due to its larger size than Ubr-box alone to increase the FP effect. Purified GST was used to ensure that the tracer does not bind to the GST portion of the fusion protein. The optimized condition includes receptor concentration [GST-UBR1]=2 μM, tracer concentration [tracer]=9 nM, and the highest concentration of unlabeled peptidomimetic compound competitors at 200 μM with 3× serial dilutions. Effective displacement of tracer by unlabeled tetrapeptide confirmed that the fluorescein tag did not significantly alter the binding mode of tracer.

Competition FP assays showed variations of the binding affinities of the peptidomimetic compounds to GST-Ubr-box (representative data shown in FIG. 4B). Preliminary ITC measurements confirmed the FP measurements (FIG. 4C). Although the Ubr-box in the Ubr family of E3 ligases is conserved, only the Ubr-boxes of Ubr1 and Ubr2 can bind to the tracer and Ubr-boxes of Ubr4 and Ubr5 showed similar results as that of GST alone (FIG. 4A), contrasting the literature that Ubr4 and Ubr5 also bind to the same N-degron as Ubr1 and Ubr2 by pull-down assays. The discrepancy likely due to that pull-down studies may reflect binding mediated by other proteins in cells, suggesting that Ubr1 and Ubr2 have different substrate specificity from other Ubr proteins.

### Example 4: Peptidomimetic Compound 4 Blocked R-GFP Degradation in Cells at >1 μM Concentration

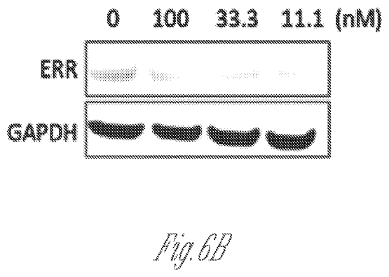

Using a model Ubr ligase substrate of an arginine (R) inserted at the N-terminus of rhacostoma green fluorescent protein (Ubiquitin-R-GFP) as described previously, expressed in cells, the peptidomimetic compound is shown to block GFP degradation, demonstrating that they penetrate cells and block Ubr-mediated GFP degradation (FIG. 5). The peptidomimetic compound outcompetes the R-GFP model Ubr substrate for E3 binding, therefore preventing GFP degradation. The upper panels show fluorescent imaging of the cells and the lower panel show a bright field of the cells.

### Example 5: Synthesis of a Peptidomimetic Compound Engaging ERRα

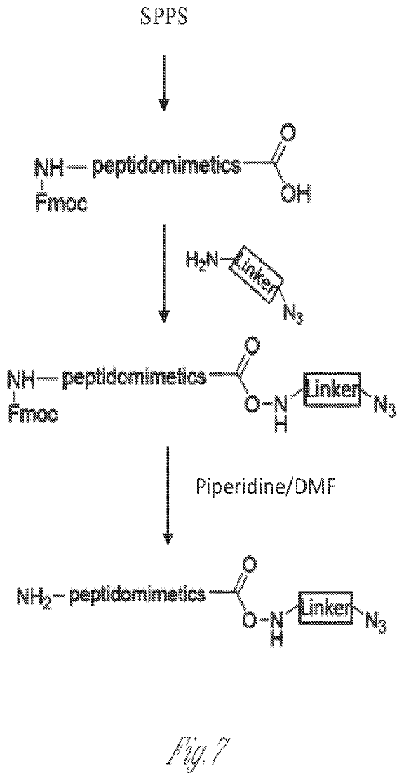

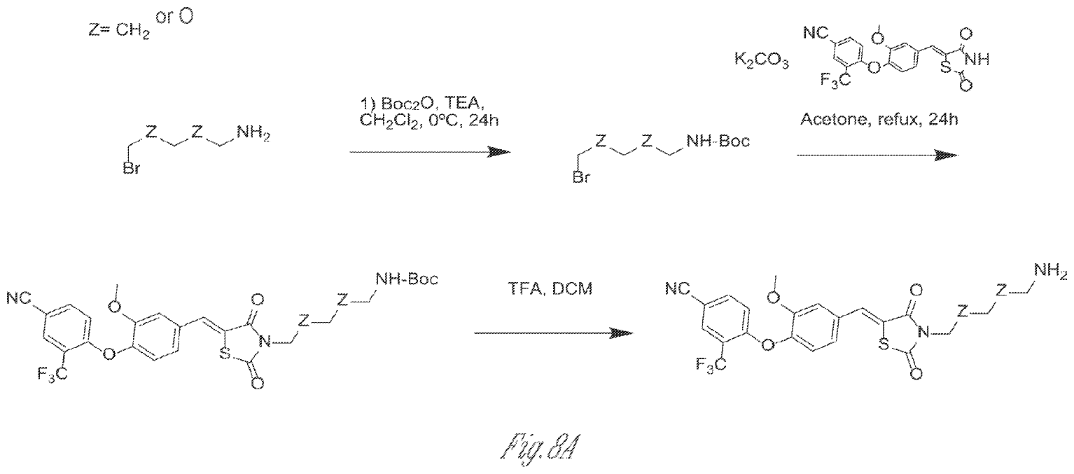

Ubr ligase engaging peptidomimetic compounds can function as a molecular glue (FIGS. 6A-B). In addition, they can be used as a platform to synthesize bifunctional small molecule to induce TDP. As an example, a peptidomimetic compound engaging Ubr and estrogen-related receptor alpha (ERRα) was synthesized using copper(I)-catalyzed azide-alkyne cycloaddition (click chemistry) (FIG. 6A). The following compound was synthesized:

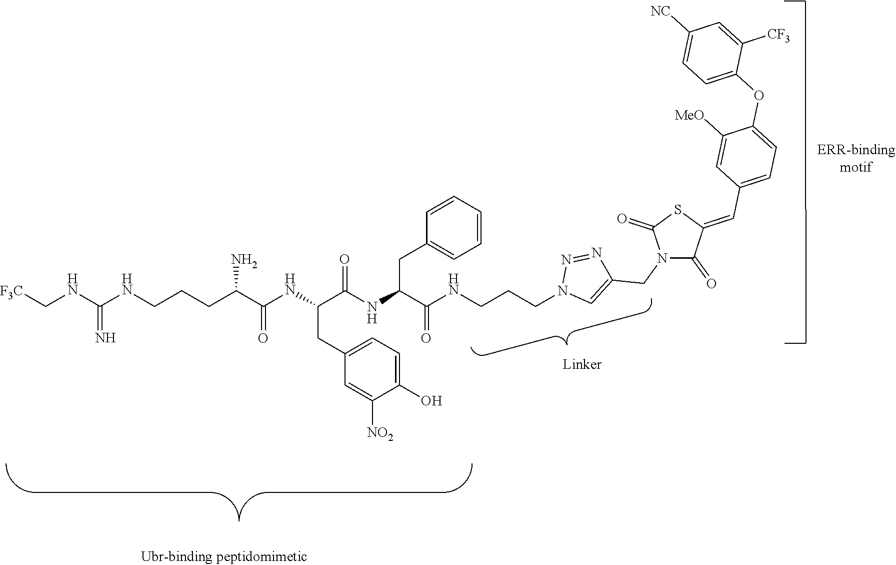 [SMILES: COc1cc(/C=C2\SC(=O)N(Cc3cn(CCCNC(=O)[C@H](Cc4ccccc4)NC(=O)[C@H](Cc4ccc(O)c([N+](=O)[O-])c4)NC(=O)[C@@H](N)CCCNC(=N)NCC(F)(F)F)nn3)C2=O)ccc1Oc1ccc(C#N)cc1C(F)(F)F]

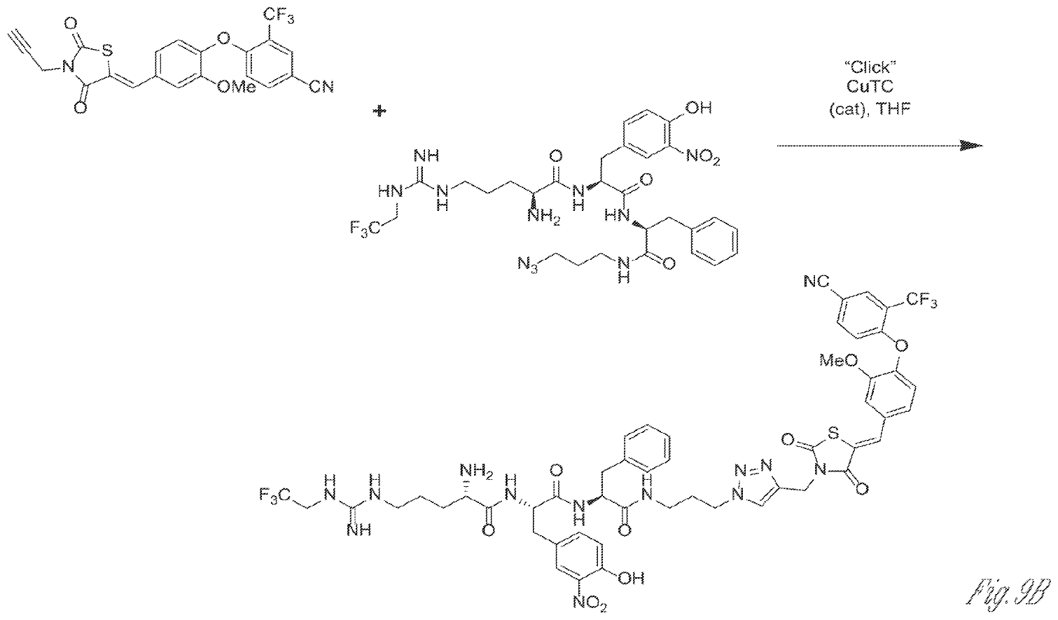

FIG. 6B is an image of a Western blot showing reduction of levels of the ERRα protein in cells upon addition of the above peptidomimetic compound (also shown in FIGS. 6A and 9). In this experiment, Hela cells were treated overnight at a concentration of 100 nM, 33.3 nM, and 11.1 nM with the above peptidomimetic compound. The ERRα protein was detected by Western blot from whole cell lysates. Glyceraldehyde 3-phosphate dehydrogenase (GAPDH) was blotted as loading control.

### Example 6: Synthesis of Peptidomimetic Compounds with Different Linkers

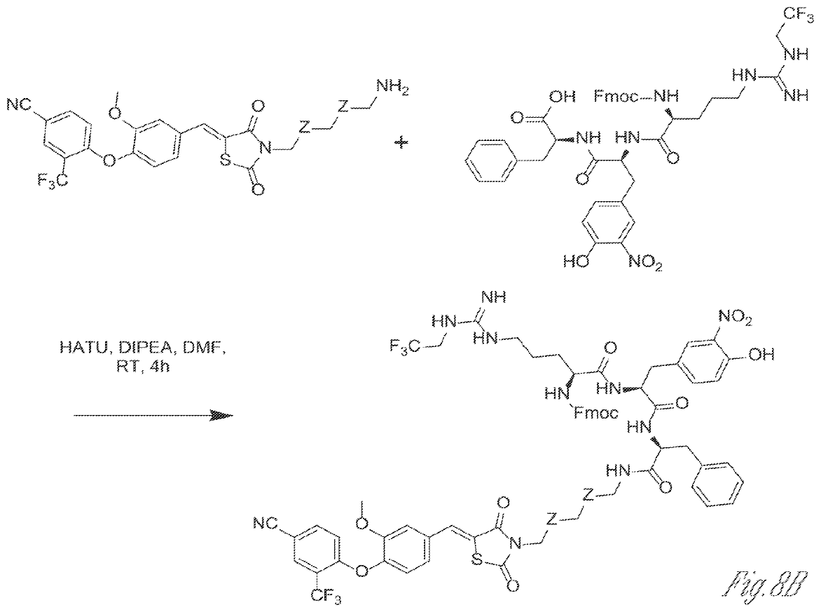

Peptidomimetic compounds with an azide linker were synthesized as shown in FIG. 7. Peptidomimetic compounds with linkers of 3 carbons, 6 carbons, or polyethylene glycol (PEG) were synthesized as shown in FIG. 8.

### Statements

The following statements are intended to describe and summarize various embodiments of the technology according to the foregoing description in the specification.
1. A compound of formulae:

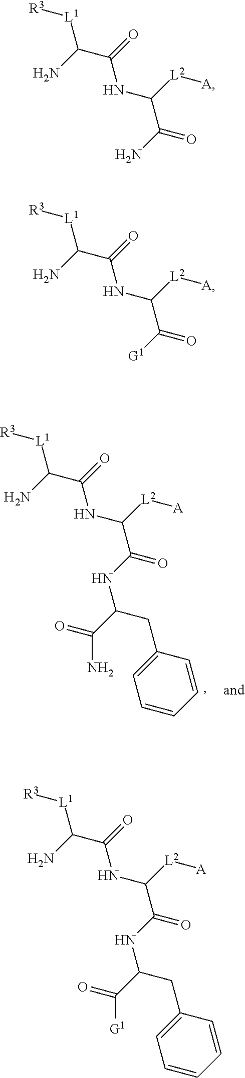

or a pharmaceutically acceptable salt thereof,
wherein:
A is an optionally substituted aryl group, an optionally substituted alkyl group or an optionally substituted heterocyclyl group;
G¹ comprises a moiety that binds to a recognition element on a target substrate protein for degradation through Ubr protein-mediated ubiquitination;
L¹ is a divalent linker;
L² is absent or a divalent alkyl linker; and
R³ comprises an ionizable group, a thiophenyl group or an amide group.
2. The compound of statement 1, wherein the compound is a compound of the formulae:

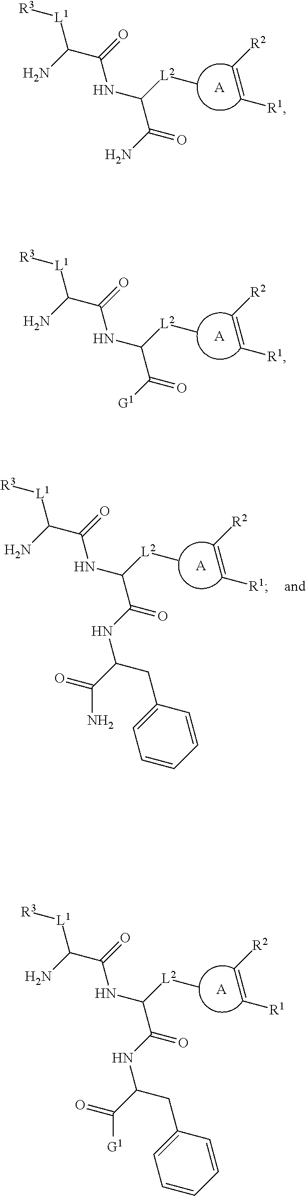

or a pharmaceutically acceptable salt thereof,
wherein:
R¹ and R² are each, independently, H or a substituent.
3. A compound of the formula:

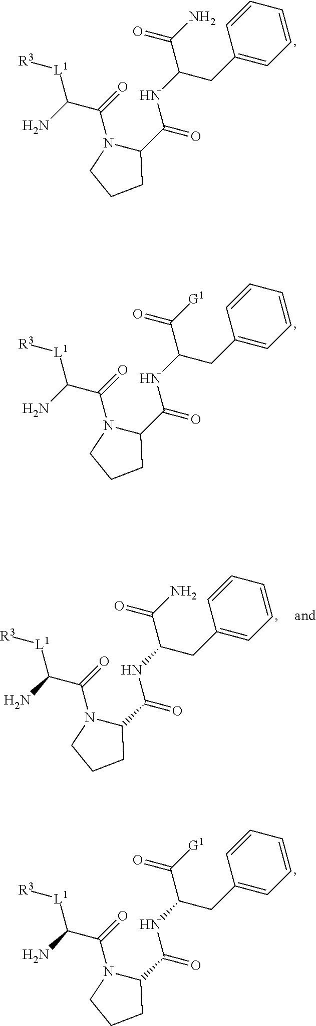

or a pharmaceutically acceptable salt thereof,
wherein:
G¹ comprises a moiety that binds to a recognition element on a target substrate protein for degradation through Ubr protein-mediated ubiquitination;
L¹ is a divalent linker;
L² is absent or a divalent alkyl linker; and
R³ comprises an ionizable group, a thiophenyl group or an amide group.
4. The compound of statement 1, 2 or 3, or a pharmaceutically acceptable salt thereof, wherein at least one of G¹ and L¹ comprises divalent C₃-C₆-alkyl group, a divalent polyethylene glycol group, one or more amino acids or combinations thereof.
5. The compound of statement 1, or a pharmaceutically acceptable salt thereof, wherein R³ comprises an amine, heterocyclyl, urea or guanidinyl groups.
6. The compound of statement 5, or a pharmaceutically acceptable salt thereof, wherein the heterocyclyl group is an imidazole, triazole, pyrrole or tetrazole.
7. The compound of statement 1, 2 or 3, or a pharmaceutically acceptable salt thereof, wherein R³ comprises a group of the formula:

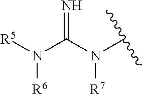

wherein:
R⁵ is H or alkyl;
R⁶ is H, alkyl or haloalkyl, such as fluoroalkyl; and
R⁷ is H or alkyl or R⁶ and R⁷, together with the atoms to which they are attached, can join to form a five- or six-membered ring.
8. The compound of statement 7, or a pharmaceutically acceptable salt thereof, wherein R³ comprises a group of the formula:

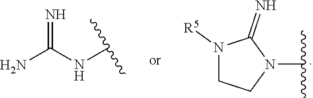

9. The compound of statement 1, or a pharmaceutically acceptable salt thereof, wherein R³-L¹ comprises a group of the formula:

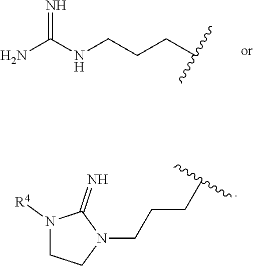

10. The compound of statement 1, 2 or 3, wherein the moiety that binds to the recognition element on a target substrate is linked directly or indirectly, by way of a divalent linker L³, to the carbonyl group to which G¹ is attached; wherein L³ is a divalent alkyl linker, an aryalkyl linking group or L¹.
11. The compound of statement 10, wherein the moiety that binds to the recognition element on the target substrate protein comprised in the group G¹ binds to the target substrate protein non-covalently or covalently.
12. The compound of statement 10, wherein the moiety that binds to the recognition element on the target substrate protein non-covalently is a moiety of the formula:

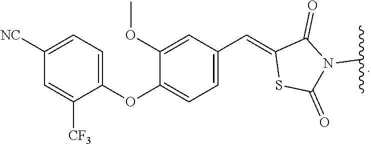 [SMILES: *N1C(=O)S/C(=C\c2ccc(Oc3ccc(C#N)cc3C(F)(F)F)c(OC)c2)C1=O]

13. A compound of the formula:

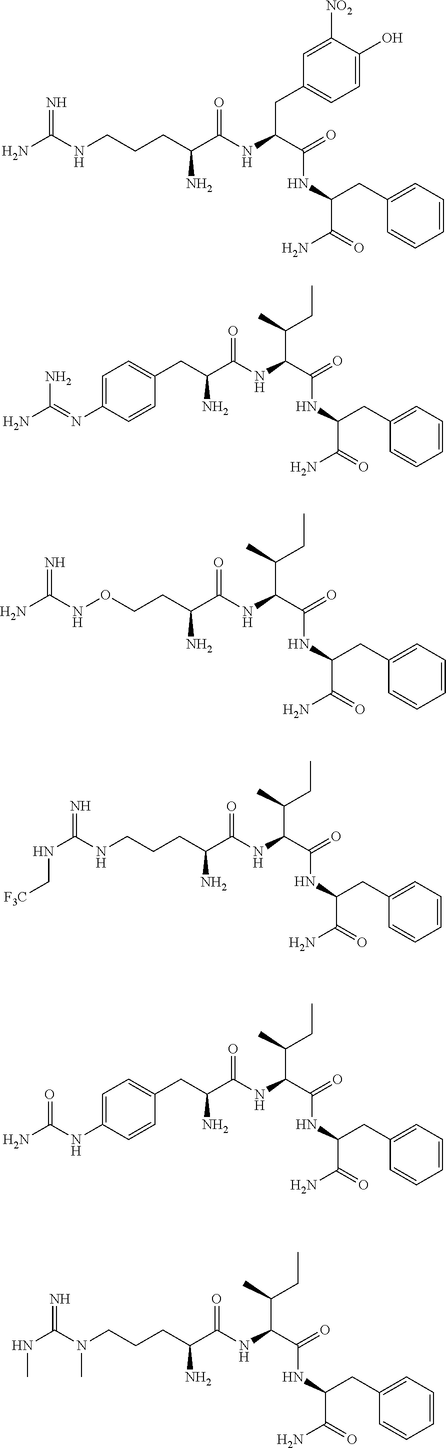

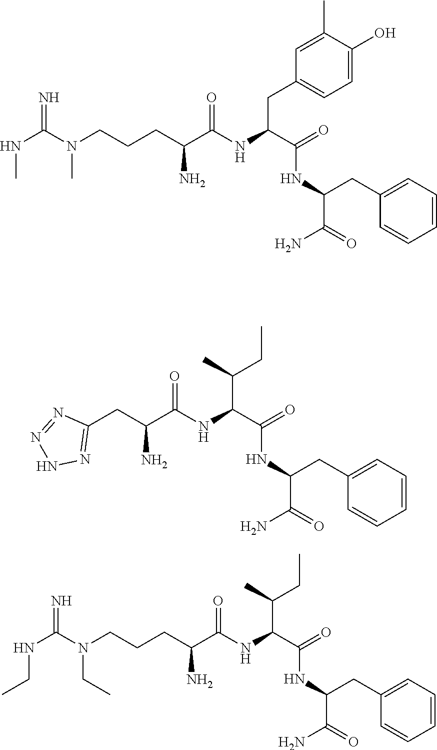

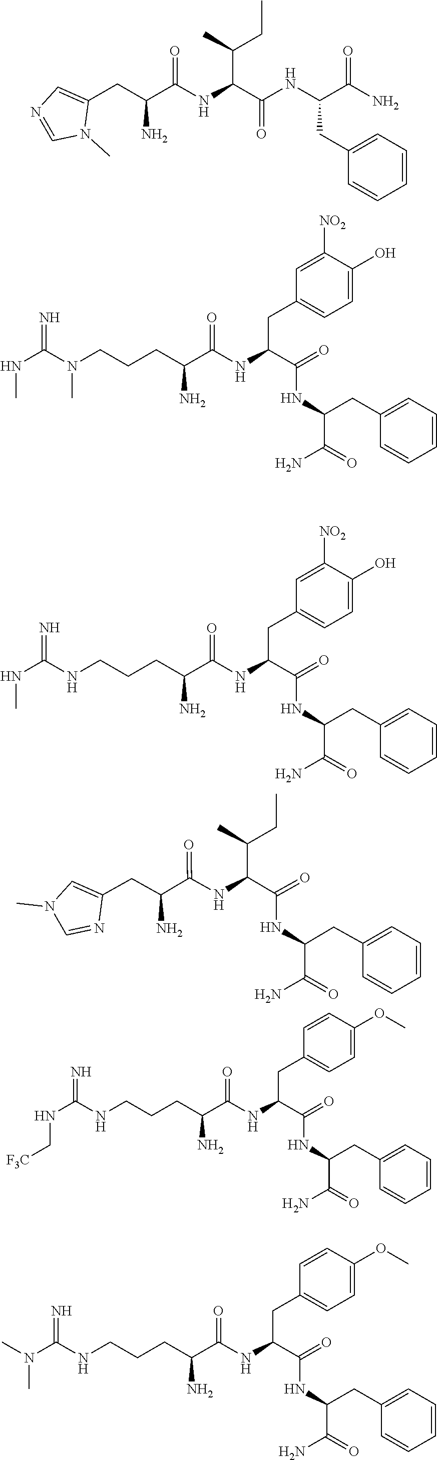

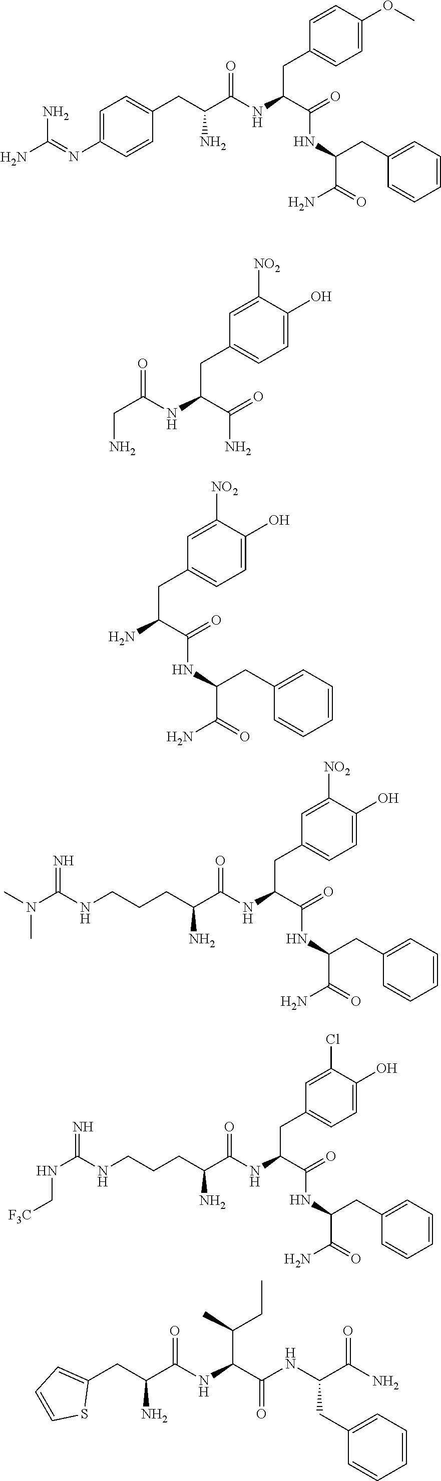

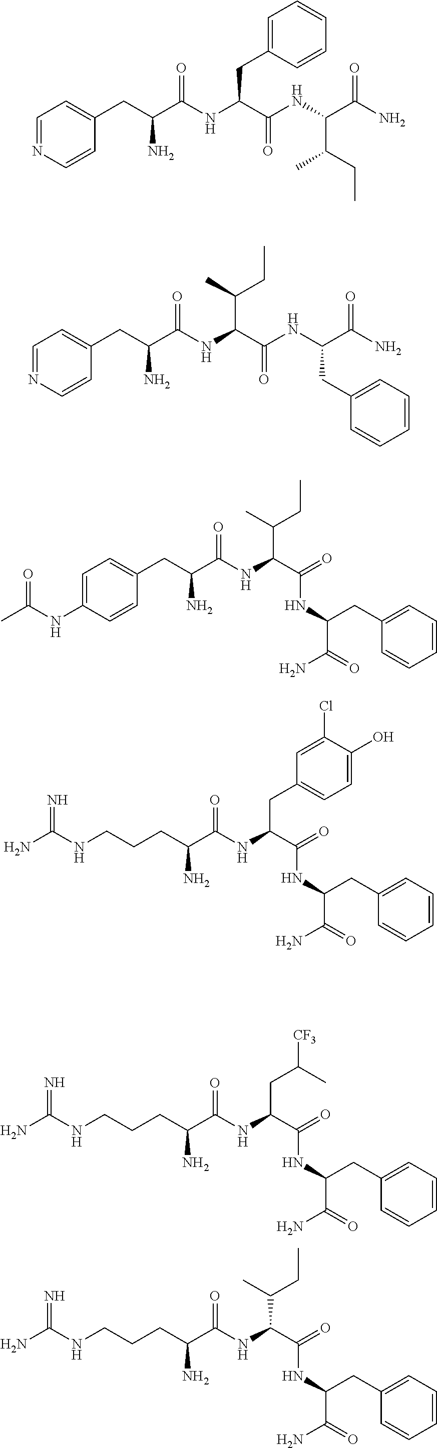

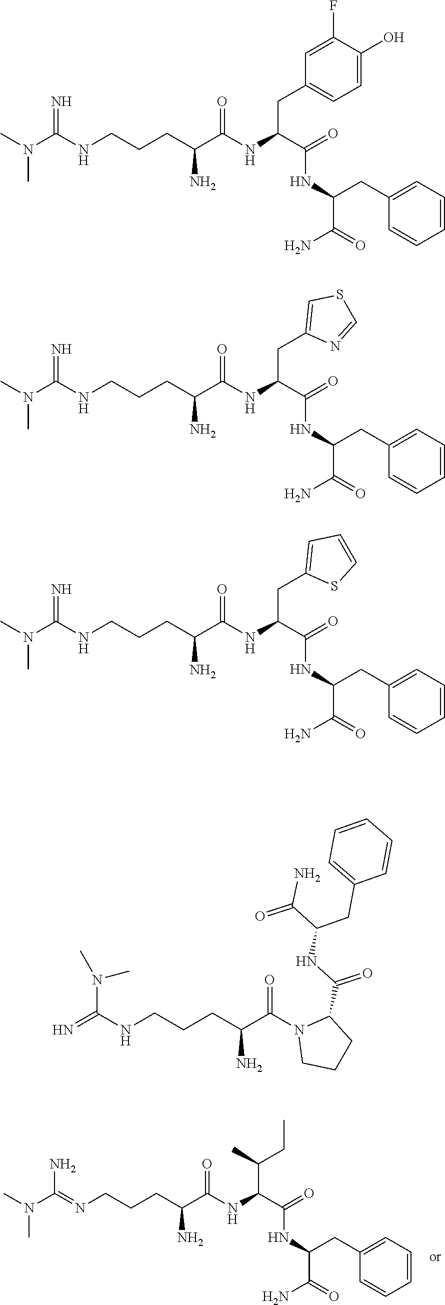 [SMILES: CN(C)C(=N)NCCC[C@H](N)C(=O)N[C@@H](Cc1ccc(O)c(F)c1)C(=O)N[C@@H](Cc1ccccc1)C(N)=O ; CN(C)C(=N)NCCC[C@H](N)C(=O)N[C@@H](Cc1cscn1)C(=O)N[C@@H](Cc1ccccc1)C(N)=O ; CN(C)C(=N)NCCC[C@H](N)C(=O)N[C@@H](Cc1cccs1)C(=O)N[C@@H](Cc1ccccc1)C(N)=O ; CN(C)C(=N)NCCC[C@H](N)C(=O)N1CCC[C@H]1C(=O)N[C@@H](Cc1ccccc1)C(N)=O ; CC[C@H](C)[C@H](NC(=O)[C@@H](N)CCC/N=C(\N)N(C)C)C(=O)N[C@@H](Cc1ccccc1)C(N)=O]

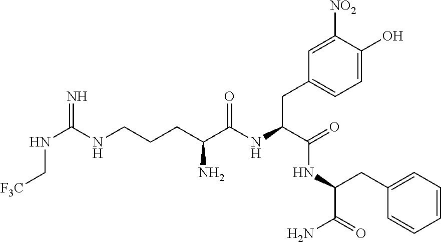 [SMILES: N=C(NCCC[C@H](N)C(=O)N[C@@H](Cc1ccc(O)c([N+](=O)[O-])c1)C(=O)N[C@@H](Cc1ccccc1)C(N)=O)NCC(F)(F)F]

14. A pharmaceutical composition comprising a therapeutically effective amount of the peptidomimetic compound of claim 1, 2 or 3, or a pharmaceutically acceptable salt thereof, and a pharmaceutically acceptable carrier.
15. A method for degrading a target substrate protein through Ubr protein-mediated ubiquitination, the method comprising administering a compound of claim 1, 2 or 3, or a pharmaceutically acceptable salt thereof, to a subject in need thereof.
16. The method of statement 15, wherein at least one of G¹ and L¹ comprises divalent C₃-C₆-alkyl group, a divalent polyethylene glycol group, one or more amino acids or combinations thereof.
17. The method of statement 15, wherein R³ comprises an amine, heterocyclyl, urea or guanidinyl groups.
18. The method of statement 17, wherein the heterocyclyl group is an imidazole, triazole, pyrrole or tetrazole.
19. The method of statement 15, wherein R³ comprises a group of the formula:

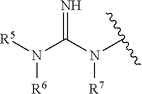

wherein:
R⁵ is H or alkyl;
R⁶ is H, alkyl or haloalkyl, such as fluoroalkyl; and
R⁷ is H or alkyl or R⁶ and R⁷, together with the atoms to which they are attached, can join to form a five- or six-membered ring.
20. The method of statement 19, wherein R³ comprises a group of the formula:

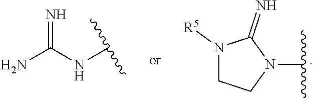

21. The method of statement 15, wherein R³-L¹ comprises a group of the formula:

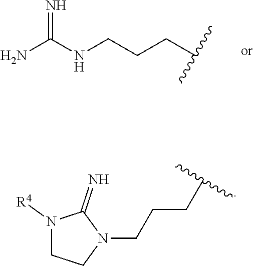

22. The method of statement 15, wherein the Ubr protein is Ubr1 or Ubr2.

The specific methods, devices and compositions described herein are representative of preferred embodiments and are exemplary and not intended as limitations on the scope of the technology. Other objects, aspects, and embodiments will occur to those skilled in the art upon consideration of this specification, and are encompassed within the spirit of the technology as defined by the scope of the claims. It will be readily apparent to one skilled in the art that varying substitutions and modifications can be made to the technology disclosed herein without departing from the scope and spirit of the technology.

The technology illustratively described herein suitably can be practiced in the absence of any element or elements, or limitation or limitations, which is not specifically disclosed herein as essential. The methods and processes illustratively described herein suitably can be practiced in differing orders of steps, and the methods and processes are not necessarily restricted to the orders of steps indicated herein or in the claims.

Under no circumstances can the patent be interpreted to be limited to the specific examples or embodiments or methods specifically disclosed herein. Under no circumstances can the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the technology as claimed. Thus, it will be understood that although the present technology has been specifically disclosed by preferred embodiments and optional features, modification and variation of the concepts herein disclosed can be resorted to by those skilled in the art, and that such modifications and variations are considered to be within the scope of this technology as defined by the appended claims and statements of the technology.

The technology has been described broadly and generically herein. Each of the narrower species and subgeneric groupings falling within the generic disclosure also form part of the technology. This includes the generic description of the technology with a proviso or negative limitation removing any subject matter from the genus, regardless of whether or not the excised material is specifically recited herein. In addition, where features or aspects of the technology are described in terms of Markush groups, those skilled in the art will recognize that the technology is also thereby described in terms of any individual member or subgroup of members of the Markush group.

1. A compound of formulae: 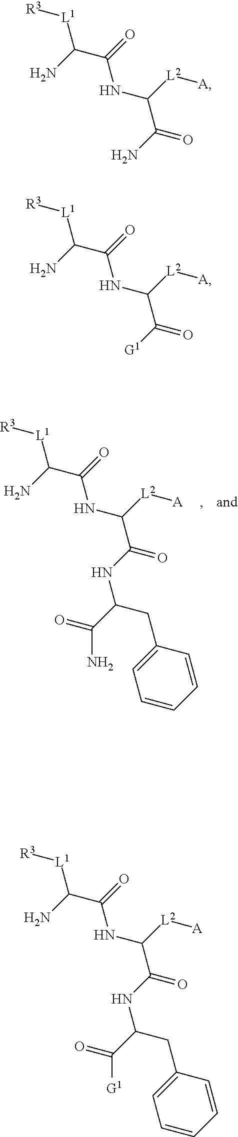
or a pharmaceutically acceptable salt thereof,
wherein:
A is an optionally substituted aryl group, an optionally substituted alkyl group or an optionally substituted heterocyclyl group;
G¹ comprises a moiety that binds to a recognition element on a target substrate protein for degradation through Ubr protein-mediated ubiquitination;
L¹ is a divalent linker;
L² is absent or a divalent alkyl linker; and
R³ comprises an ionizable group, a thiophenyl group or an amide group.

2. The compound of claim 1, wherein the compound is a compound of the formulae: 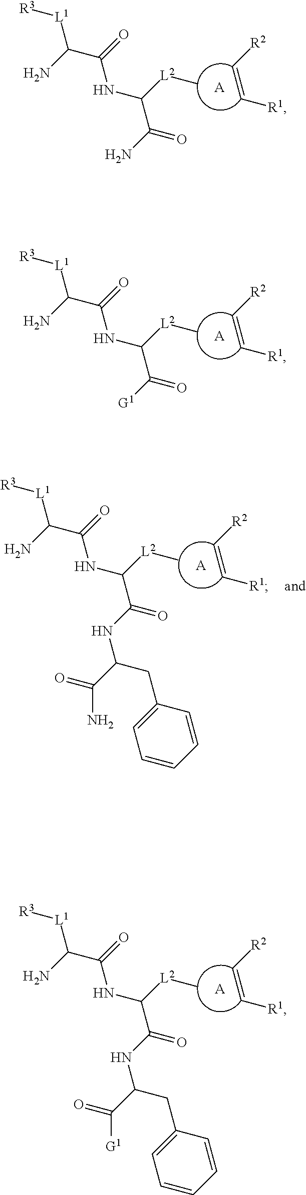
or a pharmaceutically acceptable salt thereof,
wherein:
R¹ and R² are each, independently, H or a substituent.

3. A compound of the formula: 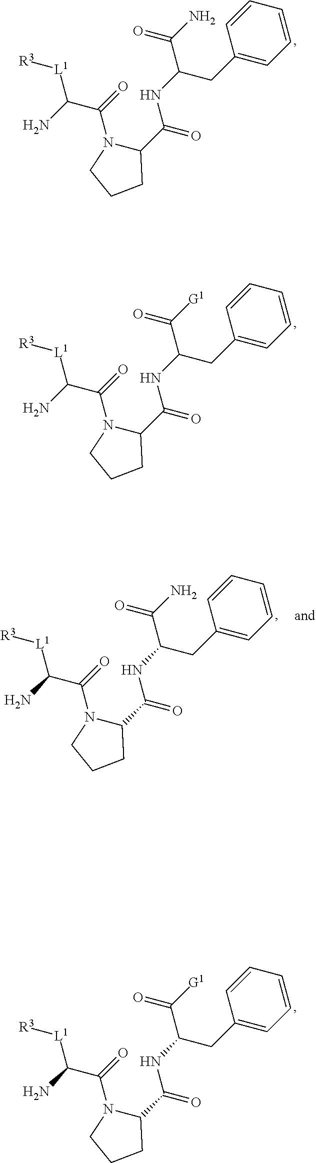
or a pharmaceutically acceptable salt thereof,
wherein:
G¹ comprises a moiety that binds to a recognition element on a target substrate protein for degradation through Ubr protein-mediated ubiquitination;
L¹ is a divalent linker;
L² is absent or a divalent alkyl linker; and
R³ comprises an ionizable group, a thiophenyl group or an amide group.

4. The compound of claim 1, or a pharmaceutically acceptable salt thereof, wherein at least one of G¹ and L¹ comprises divalent C₃-C₆-alkyl group, a divalent polyethylene glycol group, one or more amino acids or combinations thereof.

5. The compound of claim 1, or a pharmaceutically acceptable salt thereof, wherein R³ comprises an amine, heterocyclyl, urea or guanidinyl groups.

6. The compound of claim 5, or a pharmaceutically acceptable salt thereof, wherein the heterocyclyl group is an imidazole, triazole, pyrrole or tetrazole.

7. The compound of claim 1, or a pharmaceutically acceptable salt thereof, wherein R³ comprises a group of the formula: 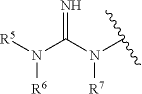
wherein:
R⁵ is H or alkyl;
R⁶ is H, alkyl or haloalkyl, such as fluoroalkyl; and
R⁷ is H or alkyl or R⁶ and R⁷, together with the atoms to which they are attached, can join to form a five- or six-membered ring.

8. The compound of claim 7, or a pharmaceutically acceptable salt thereof, wherein R³ comprises a group of the formula:

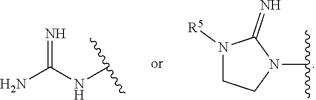

9. The compound of claim 1, or a pharmaceutically acceptable salt thereof, wherein R³-L¹ comprises a group of the formula:

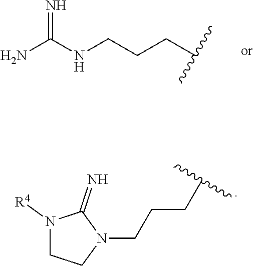

10. The compound of claim 1, [2 or 3,] wherein the moiety that binds to the recognition element on a target substrate is linked directly or indirectly, by way of a divalent linker L³, to the carbonyl group to which G¹ is attached; wherein L³ is a divalent alkyl linker, an aryalkyl linking group or L¹.

11. The compound of claim 10, wherein the moiety that binds to the recognition element on the target substrate protein comprised in the group G¹ binds to the target substrate protein non-covalently or covalently.

12. The compound of claim 10, wherein the moiety that binds to the recognition element on the target substrate protein non-covalently is a moiety of the formula:

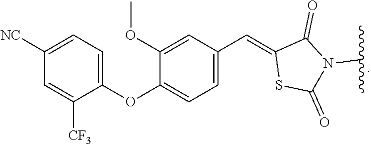 [SMILES: *N1C(=O)S/C(=C\c2ccc(Oc3ccc(C#N)cc3C(F)(F)F)c(OC)c2)C1=O]

13. A compound of the formula:

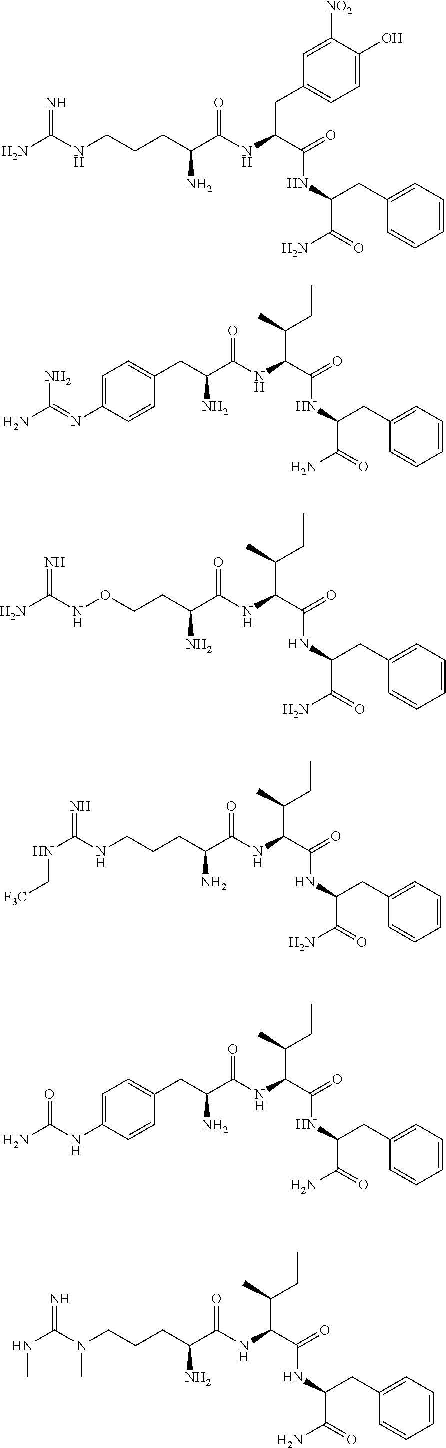

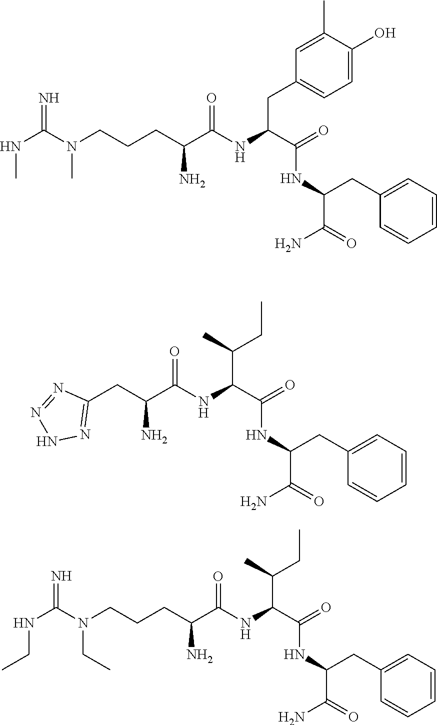

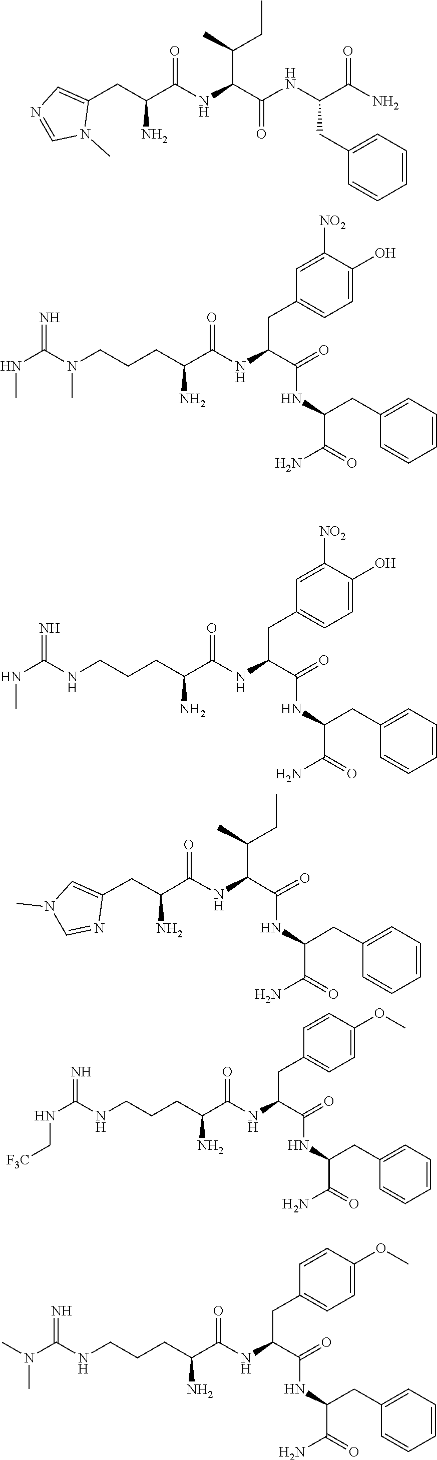

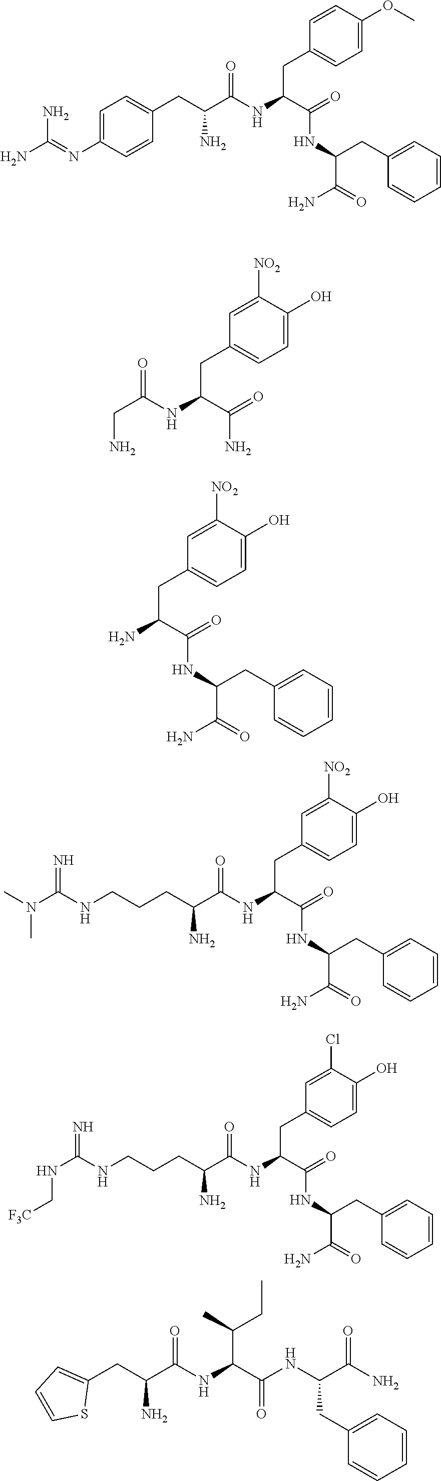

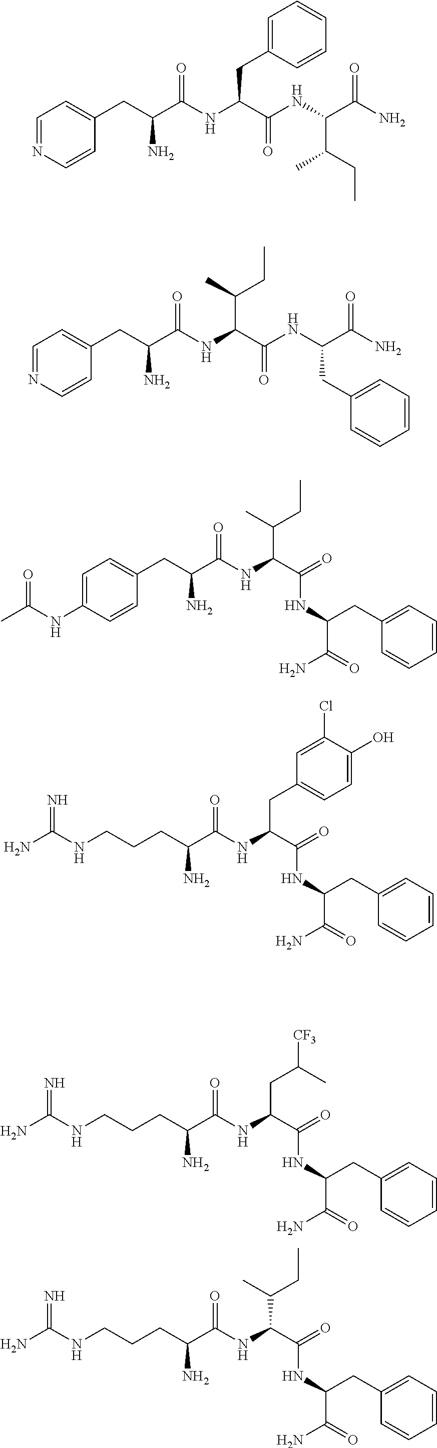

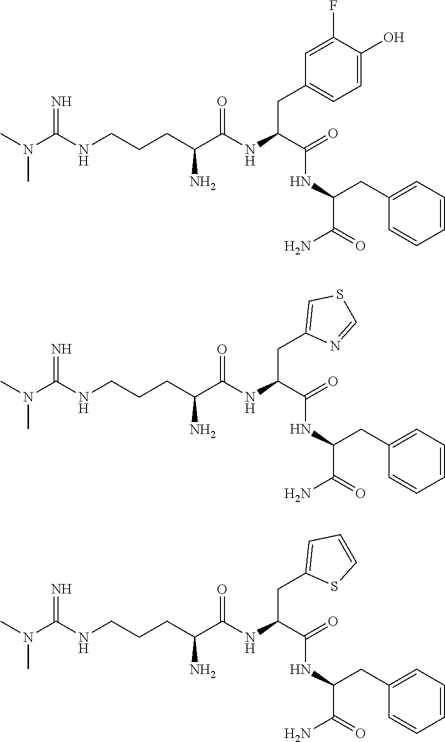 [SMILES: CN(C)C(=N)NCCC[C@H](N)C(=O)N[C@@H](Cc1ccc(O)c(F)c1)C(=O)N[C@@H](Cc1ccccc1)C(N)=O ; CN(C)C(=N)NCCC[C@H](N)C(=O)N[C@@H](Cc1cscn1)C(=O)N[C@@H](Cc1ccccc1)C(N)=O ; CN(C)C(=N)NCCC[C@H](N)C(=O)N[C@@H](Cc1cccs1)C(=O)N[C@@H](Cc1ccccc1)C(N)=O]

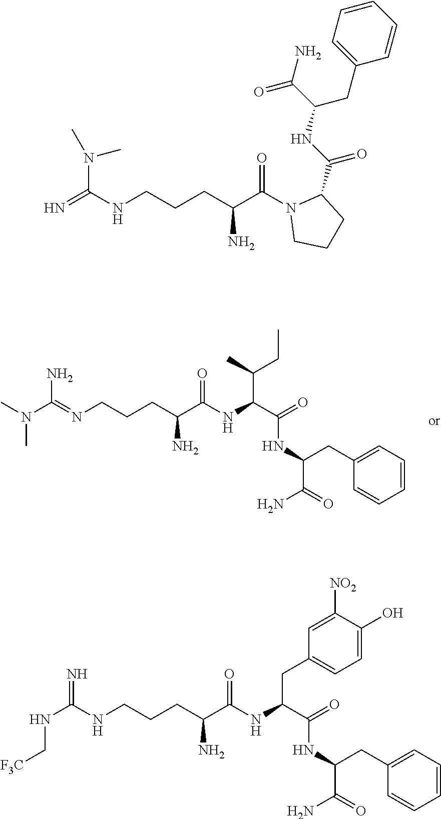 [SMILES: CN(C)C(=N)NCCC[C@H](N)C(=O)N1CCC[C@H]1C(=O)N[C@@H](Cc1ccccc1)C(N)=O ; CC[C@H](C)[C@H](NC(=O)[C@@H](N)CCC/N=C(\N)N(C)C)C(=O)N[C@@H](Cc1ccccc1)C(N)=O ; N=C(NCCC[C@H](N)C(=O)N[C@@H](Cc1ccc(O)c([N+](=O)[O-])c1)C(=O)N[C@@H](Cc1ccccc1)C(N)=O)NCC(F)(F)F]

14. A pharmaceutical composition comprising a therapeutically effective amount of the peptidomimetic compound of claim 1, or a pharmaceutically acceptable salt thereof, and a pharmaceutically acceptable carrier.

15. A method for degrading a target substrate protein through Ubr protein-mediated ubiquitination, the method comprising administering a compound of claim 1, or a pharmaceutically acceptable salt thereof, to a subject in need thereof.

16. The method of claim 15, wherein at least one of G¹ and L¹ comprises divalent C₃-C₆-alkyl group, a divalent polyethylene glycol group, one or more amino acids or combinations thereof.

17. The method of claim 15, wherein R³ comprises an amine, heterocyclyl, urea or guanidinyl groups.

18. The method of claim 17, wherein the heterocyclyl group is an imidazole, triazole, pyrrole or tetrazole.

19. The method of claim 15, wherein R³ comprises a group of the formula: 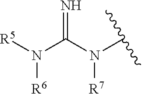
wherein:
R⁵ is H or alkyl;
R⁶ is H, alkyl or haloalkyl, such as fluoroalkyl; and
R⁷ is H or alkyl or R⁶ and R⁷, together with the atoms to which they are attached, can join to form a five- or six-membered ring.

20. The method of claim 19, wherein R³ comprises a group of the formula:

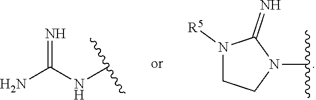

21. The method of claim 15, wherein R³-L¹ comprises a group of the formula:

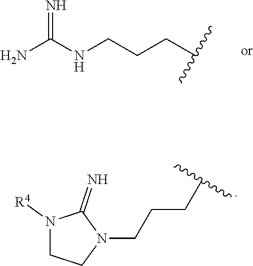

22. The method of claim 15, wherein the Ubr protein is Ubr1 or Ubr2.
